(19) United States
(12) Patent Application Publication
(10) Pub. No.: US 2023/0091231 A1
(43) Pub. Date: Mar. 23, 2023

(54) UTILIZATION OF PLANT PROTEIN HOMOLOGUES IN CULTURE MEDIA

(71) Applicants: Yissum Research Development Company of the Hebrew University of Jerusalem Ltd., Jerusalem (IL); Future Meat Technologies Ltd., Rehovot (IL)

(72) Inventors: Yaakov Nahmias, Mevaseret Zion (IL); Merav Cohen, Jerusalem (IL); Jonathan Caspi, Jerusalem (IL)

(73) Assignees: Yissum Research Development Company of the Hebrew University of Jerusalem Ltd., Jerusalem (IL); Future Meat Technologies Ltd., Rehovot (IL)

(21) Appl. No.: 17/794,172

(22) PCT Filed: Jan. 20, 2021
(86) PCT No.: PCT/IB2021/050413

§ 371 Date: Jul. 20, 2022

Related U.S. Application Data
(60) Provisional application No. 62/963,808, filed on Jan. 21, 2020.

(51) Int. Cl. C12N 5/077; C12N 9/08; A23L 13/00

(52) U.S. Cl. CPC C12N 5/0656; C12N 9/0065; A23L 13/00; C12N 2500/92; C12N 2533/52; C12N 2501/33; C12N 2500/76; A23V 2002/00

(57) ABSTRACT

The present disclosure provides, in part, a cell culture medium supplement comprising at least one plant protein homologue of a serum protein, a cell culture medium comprising a serum-free base medium and one or more plant based proteins, and methods of growing cells in vitro and of producing cultured meat using the cell culture medium.

### CROSS-REFERENCE TO RELATED APPLICATIONS

This application claims the benefit of priority to U.S. Provisional Patent Application No. 62/963,808, filed on Jan. 21, 2020, which is incorporated herein by reference in its entirety.

### SEQUENCE LISTING

The ASCII file, entitled 108912-675980_SL.txt, created on Jan. 19, 2021, comprising 55,930 bytes, submitted concurrently with the filing of this application is incorporated herein by reference. The Sequence Listing submitted herewith is identical to the Sequence Listing forming part of the application.

### FIELD OF THE INVENTION

The present invention generally relates to cell growth. More specifically, the present invention relates to cell growth media essentially devoid of animal serum-derived components and methods of growing cells in the media and thereby producing cultured meat.

### BACKGROUND

The current world population is over 7 billion and still rapidly growing. In order to support the nutritional requirement of this growing population, an increasing amount of land is dedicated for food production. The natural sources are insufficient to fulfill the demand. This has led to famine in some parts of the world. In other parts of the world, the problem is being addressed by large-scale production of animals in dense factory farms under harsh conditions. This large-scale production not only causes great suffering to animals, but also increases arsenic levels and drug resistance bacteria in meat products due to organoarsenic compounds and antibiotics used to increase food efficiency and control infection, thus further increases the number of diseases and worsens the consequences thereof for both animals and humans. Large-scale slaughtering is required to fulfill the current food requirements and as a consequence, it can lead to large-scale disease outbreaks such as the occurrence of porcine pestivirus and mad cow disease. These diseases result in loss of the meat for human consumption thus completely denying the purpose for which the animals were being bred in the first place.

In addition, the large-scale production reduces the flavor of the finished product. A preference exists among those that can afford non-battery laid eggs and non-battery produced meat. It is not only a matter of taste, but also a healthier choice thereby avoiding consumption of various feed additives such as growth hormones. Another problem associated with mass animal production is the environmental problem caused by the vast amounts of fecal matter from the animals and which the environment subsequently has to deal with. Moreover, the large amount of land currently required for the production of animals or the feed for the animals which cannot be used for alternative purposes such as growth of other crops, housing, recreation, wild nature and forests is problematic.

One of the primary problems of the techniques known in the art is that, with a long time to produce, and at extremely high costs, products are of a mediocre quality that cannot and will not replace the current meat derived from livestock. For example, Just-Inc. grows extracted animal cells in media to manufacture chicken nuggets, which cost $50 per nugget to manufacture.

Culture of cells, e.g., mammalian cells or insect cells, for in vitro experiments or ex vivo culture, for administration to a human or animal is an important tool for studies and treatments of human diseases. Cell culture is widely used for the production of various biologically active products, e.g., viral vaccines, monoclonal antibodies, polypeptide growth factors, hormones, enzymes, tumor specific antigens and food products. However, many of the media or methods used to culture the cells comprise components that can have negative effects on cell growth and/or maintenance of an undifferentiated cell culture. For example, mammalian or insect cell culture media is often supplemented with blood-derived serum such as fetal calf serum (FCS) or fetal bovine serum (FBS) in order to provide growth factors, carrier proteins, attachment and spreading factors, nutrients and trace elements that promote proliferation and growth of cells in culture. However, the factors found in FCS or FBS, such as transforming growth factor (TGF) beta or retinoic acid, can promote differentiation of certain cell types (Ke et al., Am J Pathol. 137: 833-43, 1990) or initiate unintended downstream signaling in the cells that promotes unwanted cellular activity in culture (Veldhoen et al., Nat Immunol. 7(11): 1151-6, 2006).

The cost of culture medium is the primary driving factor of the cost of cultured meat production. Culture medium is composed of relatively simple basal medium that comprises carbohydrates, amino acids, vitamins and minerals and much more expensive serum replacement component including; albumin, growth factors, enzymes, attachment factors and hormones. In order to eliminate the use of animal components, industry is currently relying on recombinant human proteins for applications in cell therapy and vaccine production. However, cultured meat applications are not limited to the use of human proteins, thus can potentially utilize a more readily available source of materials that is suitable for human consumption.

The uncharacterized nature of the serum composition and lot-to-lot variation of the serum make use of a serum replacement and culture in serum-free media desirable (Pei et al., Arch Androl. 49(5): 331-42, 2003). Moreover, for cells, recombinant proteins or vaccines for therapeutic use that are grown in cell culture, the addition of animal-derived components is undesirable due to potential virus contamination and/or to the potential immunogenic effect of the animal proteins when administered to humans.

Serum replacements have been developed in attempts to minimize the effects of FCS on cell culture, as well as minimize the amount of animal proteins used for culturing human cells. Serum replacement, such as KNOCKOUT™ serum replacement (Invitrogen, Carlsbad, Calif.), a chemically defined culture medium lacking serum and containing essential nutrients and other proteins for cell growth. KNOCKOUT™ cannot be used as a replacement for FBS in the plating of feeder cells due to the lack of attachment factors, which results in inadequate cell attachment in this formulation. PC-1™ serum free media (Lonza, Walkersville, Md.) is a low-protein, serum-free medium formulated in a specially modified DMEM/F12 media base and contains a complete HEPES buffering system with known amounts of insulin, transferrin, fatty acids and proprietary proteins.

Cellgro COMPLETE™ (Cellgro, Manassas, Va.) is a serum-free, low-protein culture media based on a mix of DMEM/F12, RPMI 1640 and McCoy's 5A base mediums. Cellgro COMPLETE™ does not contain insulin, transferrin, cholesterol, growth or attachment factors, but comprises a mixture of trace elements and high molecular weight carbohydrates, extra vitamins, a non-animal protein source, and bovine serum albumin.

Recombinant protein produced in animal cells or plants are currently used in culture media. For example, recombinant human albumin is produced in rice, while recombinant fibronectin is produced in mouse cells. There is a need for medium supplements without the undesirable side effects of animal products or recombinant protein production for growth or attachment factor serum components. The present invention fulfills this long-standing need.

### SUMMARY OF THE INVENTION

The cost of culture medium is the primary driver of the cost of cultured meat production. Culture medium is composed of relatively simple basal medium containing; carbohydrates, amino acids, vitamins and minerals and much more expensive serum replacement component including; albumin, growth factors, enzymes, attachment factors and hormones. In order to eliminate the use of animal components, industry is currently relying on recombinant human proteins for applications in cell therapy and vaccine production. However, cultured meat applications are not limited to the use of human proteins, thus can potentially utilize a more readily available source of materials that is suitable for human consumption.

The present disclosure is based, in part, on the finding that replacements for some of the most expensive components of serum can be found in protein-homologues in the plant kingdom. For example, plant albumins and globulins can surprisingly replace serum albumin as lipid and growth factor carriers in culture media. Catalase is an important enzyme in animal serum to remove hydrogen peroxide and is also abundant in potatoes, cucumbers and other plants. Homologs to common attachment factors, such as fibronectin and vitronectin can be found between plant cell walls and their membranes. The use of such plant-based proteins in culture media significantly reduces the cost of the medium for the production of cultured meat.

One aspect of the present disclosure provides a cell culture medium supplement comprising at least one at least one plant protein homologue of a serum protein.

In some embodiments the cell culture medium supplement is devoid of any serum proteins. In some embodiments the cell culture medium supplement is essentially devoid of any animal serum-derived components.

In some embodiments, the at least one plant protein homologue comprises the water soluble fraction of a plant protein isolate. In some embodiments, the water soluble fraction comprises plant albumins and globulins.

In some embodiments, the at least one plant protein homologue is a homologue of a serum albumin, a serum catalase, a serum superoxide dismutase, a serum transferrin, a serum fibronectin, a serum vitronectin, a serum insulin, a serum hemoglobin, a serum aldolase, a serum lipase, a serum transaminase, a serum aminotransferase, a serum fetuin, or a combination thereof.

In some embodiments, the at least one plant protein homologue is a plant albumin, a plant catalase, a plant superoxide dismutase, a plant transferrin, a plant fibronectin, a plant vitronectin, a plant insulin, a plant leghemoglobin, a plant aldolase, a plant lipase, a plant transaminase, a plant aminotransferase, a plant cystatin, or a combination thereof.

In some embodiments, the supplement comprises a plant albumin, a plant catalase, a plant fibronectin, and a plant insulin.

In some embodiments, the supplement further comprises a plant transferrin.

In some embodiments, the supplement further comprises a plant superoxide dismutase.

In some embodiments, the supplement further comprises a plant vitronectin.

In some embodiments, the at least one plant protein homologue is a plant albumin. In some embodiments, the plant albumin is a chickpea albumin, a hempseed albumin, a lentil albumin, a pea albumin, a soy albumin, a wheat albumin or a potato albumin. In some embodiments, the plant albumin is a pea albumin or a potato albumin.

In some embodiments, the plant albumin is from the water soluble fraction of a plant protein isolate.

In some embodiments, the plant albumin has a molecular weight of about 13-110 kilodaltons. In some embodiments, the plant albumin has a molecular weight of about 13-17 kilodalton. In some embodiments, plant albumin has a molecular weight of about 20-35 kilodalton. In some embodiments, plant albumin has a molecular weight of about 50-110 kilodalton.

In some embodiments, the plant albumin is present at a concentration in the cell culture medium supplement such that the plant albumin has a final concentration of about 0.01% to about 10% by weight in the cell culture medium.

In some embodiments, the at least one plant protein homologue is a plant catalase.

In some embodiments, the plant catalase is an Arabidopsis catalase, a cabbage catalase, a cucumber catalase, a cotton catalase, a potato catalase, a pumpkin catalase, a spinach catalase, a sunflower catalase, a tobacco catalase or a tomato catalase. In some embodiments, the plant catalase is a cabbage catalase, a cucumber catalase or a potato catalase.

In some embodiments, plant catalase has a molecular weight of about 50-70 kilodaltons.

In some embodiments, plant catalase is present in the cell culture medium supplement at a concentration such that when the cell culture medium supplement is added to a cell culture medium the plant catalase has a final concentration of about 1 ng/ml to about 100 ng/ml in the cell culture medium.

In some embodiments, the at least one plant protein homologue is a plant fibronectin.

In some embodiments, the plant fibronectin is a bean fibronectin, a chickpea fibronectin, a lentil fibronectin, a rice fibronectin, a soy fibronectin, a tobacco fibronectin or a wheat fibronectin. In some embodiments, the plant fibronectin is a chickpea fibronectin, a lentil fibronectin, a rice fibronectin, a soy fibronectin or a wheat fibronectin.

In an embodiment, said plant fibronectin has a molecular weight of about 40-60 kilodaltons.

In some embodiments, the plant fibronectin has a final concentration of about 0.1 μg/ml to about 100 μg/ml in the cell culture medium.

In some embodiments, the at least one plant protein homologue is a plant insulin.

In some embodiments, the plant insulin is glucokinin, charantin, or corosolic acid.

In some embodiments, the plant insulin has a final concentration of about 0.05 μg/ml to about 10 μg/ml in the cell culture medium.

In some embodiments, the at least one plant protein homologue is a plant transferrin.

In some embodiments, the at least one plant protein homologue is a plant vitronectin.

In some embodiments, the at least one plant protein homologue is a plant superoxide dismutase.

In some embodiments, the at least one plant protein homologue is in the form of plant extract fraction or a pure form.

Still another aspect of the present disclosure provides a cell culture medium comprising a serum-free medium and any of the herein disclosed cell culture medium supplements.

In some embodiments, the cell culture medium is devoid of any animal serum proteins.

In some embodiments, the cell culture medium is essentially devoid of any animal serum-derived components.

In some embodiments, the serum-free medium is a base medium. In some embodiments, the base medium is a base physiological buffer.

Still another aspect of the present disclosure provides a kit comprising any of the herein disclosed cell culture medium supplements and instructions for mixing the supplement with a serum-free medium devoid of any animal components and/or animal proteins.

Yet another aspect of the present disclosure provides a method of producing cultured meat by culturing cells in any of the herein disclosed cell culture medium and producing meat from the cultured cells.

In some embodiments, the cells are from edible animals. In some embodiments, the edible animal is livestock, game, poultry, fish, or crustacean.

In some embodiments, the method comprises cultured cells wherein the cells are fibroblasts. In an embodiment the fibroblasts are bovine fibroblasts or chicken fibroblasts.

Yet another aspect of the present disclosure provides cultured meat produced by the methods disclosed above and herein.

Still yet another aspect of the present disclosure provides a method for producing a cell culture medium devoid of any animal proteins and/or animal components. The method comprises admixing a serum-free base medium essentially devoid of any animal serum-derived components and any of the herein disclosed cell culture medium supplements essentially devoid of any animal serum-derived components.

Still yet another aspect of the present disclosure provides a cell culture medium produced by the above method.

Further aspect of the present disclosure provides use of a plant protein homologue of an animal protein in place of the animal protein in a cell culture medium supplement. In an embodiment, the animal protein is a serum protein. In some embodiments, the supplement is essentially devoid of any animal serum-derived components.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and other features of the invention are explained in the following description, taken in connection with the accompanying drawings, wherein:

FIG. 1 depicts an alignment of legume albumin homologues of serum albumin (SEQ ID NOs: 1-13).

FIG. 2 depicts an alignment of seed storage albumin homologues of serum albumin (SEQ ID NOs: 14-43).

FIGS. 3A-3E depict mass-spectrometry (MS) analysis of extracted potatoes. FIG. 3F depicts SDS-PAGE analysis of extracted potatoes.

FIG. 4 depicts SDS-PAGE analysis of pea protein.

FIG. 5 depicts SDS-PAGE analysis of water soluble protein fractions of five plant flours (durum, chickpea, lentil, corn, rice) and two commercial plant protein isolates (hemp, pea).

FIG. 6 depicts results of MS analysis of four potato extractions from two potato types (Red or White).

FIG. 7 depicts attachment in soy, chickpea, lentil, rice and wheat extracts in cultured cells in the absence of serum and animal-derived ECM proteins.

FIG. 8 is a schematic diagram showing preparation of complete protein bulks as a replacement of bovine serum albumin (BSA).

FIG. 9 depicts SDS-PAGE analysis of soy protein (water soluble fraction) before and after Albusorb purification.

FIG. 10A depicts MS analysis of top 10 water soluble soy protein groups. FIG. 10B depicts MS analysis of top 10 Albusorb purified soy protein groups.

FIG. 11 depicts mass-spectrometry (MS) analysis of chickpea proteins.

FIG. 12 depicts effect of different plant water soluble fraction proteins on chicken fibroblast cells using a special serum free supplement devoid of BSA proteins.

FIG. 13 depicts dose dependent effect of both chickpea and organic pea proteins on chicken fibroblasts in a suspension culture to replace BSA in a serum free medium.

FIG. 14 depicts dose dependent toxicity of chickpea protein.

FIG. 15 depicts chickpea protein optimization for cell growth.

### DETAILED DESCRIPTION OF THE INVENTION

For the purposes of promoting an understanding of the principles of the present disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The terminology used herein is for the purpose of describing particular embodiments only and is not intended to be limiting of the invention.

As used herein, the term “animal component” or “animal components” refers to a composition in which the components are derived, obtained, sourced, or produced from animals. The components are not “animal components” if they are produced recombinantly or derived from plants or sources other than an animal. As used herein, “animal component” does not include recombinant production of components of the media in cell lines, including recombinant animal components. Nor does “animal component” include components produced in animal cell lines.

As used herein, the term “animal serum-derived component” or “animal serum-derived components” refers to a composition in which the components are derived, obtained, sourced, or produced from animal serum. The components are not “animal serum-derived components” if they are produced recombinantly or derived from plants or sources other than an animal serum. As used herein, “animal serum-derived component” does not include recombinant production of components of the media in cell lines, including recombinant animal components. Nor does “animal serum-derived component” include components produced in animal cell lines.

As used herein “devoid of” or “free” (as in “animal component free”), “essentially devoid of” or “essentially free”, means non-detectable or a small or insignificant amount of a contaminant. The term “non-detectable” is understood as based on standard methodologies of detection known in the art at the time of this application. In some embodiments, “a small amount” refers to less than 1% by weight.

As used herein, “animal component free”, “devoid of animal components”, or “essentially devoid of animal components” refers to a composition in which the components are not derived, obtained, sourced, or produced from animals. It is contemplated that the components are either produced recombinantly or derived from plants or sources other than an animal. As used herein, “animal component free”, “devoid of animal components”, or “essentially devoid of” allows for recombinant production of components of the media in animal-based cell lines.

As used herein, the term “basal media”, “basal medium”, “base media”, “base medium”, “base nutritive medium”, or “base nutritive media” refers to a basal salt nutrient(s) or an aqueous solution(s) of salts and other elements that provide cells with water and certain bulk inorganic ions essential for normal cell metabolism and maintains intra- and extra-cellular osmotic balance. In some embodiments, a base medium comprises at least one carbohydrate as an energy source, and/or a buffering system to maintain the medium within the physiological pH range. Examples of commercially available basal media include, but are not limited to, phosphate buffered saline (PBS), Dulbecco's Modified Eagle's Medium (DMEM), Minimal Essential Medium (MEM), Basal Medium Eagle (BME), RPMI 1640, Ham's F-10, Ham's F-12, α-Minimal Essential Medium (αMEM), Glasgow's Minimal Essential Medium (G-MEM), Iscove's Modified Dulbecco's Medium, or general purpose media modified for use with pluripotent cells, such as X-VIVO (Lonza) or a hematopoietic base media.

As used herein, a “B27 supplement” also known as a “B22 supplement”) is a medium supplement that contains 21 components and 100 g BSA (such as a fraction V IgG free fatty acid poor Invitrogen 30036578×1 unit), assembled in, for example, a Neurobasal medium (Invitrogen 21103-049×2 units). The following 21 components are present in the B27 supplement: 1) Catalase, 2) Glutathione reduced, 3) Human Insulin, 4) Superoxide Dismutase (SOD), 5) Human Holo-Transferrin, 6) T3, 7) L-carnitine, 8) Ethanolamine, 9) D+-galactose, 10) Putrescine, 11) Sodium selenite, 12) Corticosterone, 13) Linoleic acid, 14) Linolenic acid, 15) Progesterone, 16) Retinol acetate, 17) DL-alpha tocopherol (vit E), 18) DL-alpha tocopherol acetate, 19) Oleic acid, 20) Pipecolic acid and 21) Biotin. The B27 supplement may be modified as a Vitamin A free B27 supplement: remove Retinol acetate, a T3 free B27 supplement: T3 (#6) can be omitted when relevant, an Anti-oxidant (AO) free B27 supplement: the following five antioxidants: #1, #2, #4, #17, #18 should be omitted and a BSA Free B27 supplement: Eliminate BSA. If needed, Human Recombinant Albumin or Serum albumin can be used instead.

As used herein, a “complete medium” refers to a basal medium further comprising added supplements, such as growth factors, hormones, proteins, serum or serum replacement, trace elements, sugars, antibiotics, antioxidants, etc., that can contribute to cell growth. For example, a commercially available complete medium comprises supplements such as ethanolamine, glutathione (reduced), ascorbic acid phosphate, insulin, human transferrin, a lipid-rich bovine serum albumin, trace salts, sodium selenite, ammonium matavanadate, cupric sulfate and manganous chloride (DMEM ADVANCED™ Media, Life Technologies).

As used herein, the term “connective tissue cells” refers to the various cell types that make up connective tissue. For example, connective tissue cells are fibroblasts, cartilage cells, bone cells, fat cells and smooth muscle cells, or a cell type that can be naturally differentiated from a fibroblast. As used herein, the term “natural differentiation” or “naturally differentiated from” is used to refer to a differentiation that occurs in nature and not a trans-differentiation such as one that can be artificially achieved in a laboratory and is not dedifferentiation. A cell type that can be naturally differentiated from a fibroblast includes a chondrocyte, an adipocyte, an osteoblast, an osteocyte, a myofibroblast, a myoblast and a myocyte. Connective tissue cells are not mesenchymal stem cells (MSCs) or cells derived from MSCs or pluripotent cells.

As used herein, the phrase “spontaneously immortalized fibroblast” refers to a fibroblast cell which is capable of undergoing unlimited cell division, and preferably also cell expansion, without being subjected to man-induced mutation, e.g., genetic manipulation, causing the immortalization. The spontaneously immortalized fibroblast is non-genetically modified.

As used herein a “liquid base mix” or “base physiological buffer liquid mix” refers to the base liquid solution of the serum replacement or media supplement into which the liposomes are suspended to complete the cell culture media composition. It is contemplated that the liquid base mix is loaded into the liposomes such that the liposome delivers an amount of the liquid base mix to cells when fused to/taken up by cells in cell culture. It is also contemplated herein that the liquid base mix or base physiological buffer liquid mix is a base medium, a complete medium or a physiological buffer solution, such as phosphate buffered saline (PBS) and other balanced salt solutions, which can be used in conjunction with the liposomes and/or other components herein to form a serum replacement, a complete medium, a medium supplement, or a cryopreservation medium.

As used herein, a “medium” or “cell culture medium” refers to an aqueous based solution that provides for the growth, viability, or storage of cells. A medium as contemplated herein can be supplemented with one or more nutrients to promote the desired cellular activity, such as cell viability, growth, proliferation, differentiation of the cells cultured in the medium. A medium, as used herein, includes a serum replacement, a medium supplement, a complete medium or a cryopreservation medium. The pH of a culture medium should be suitable to the microorganisms that will be grown. Most bacteria grow in pH 6.5-7.0 while most animal cells thrive in pH 7.2-7.4.

As used herein, a “medium supplement” refers to an agent or composition that is added to base medium prior to culture of cells. A medium supplement can be an agent that is beneficial to cell growth in culture, such as growth factor(s), hormone(s), protein(s), serum or serum replacement, trace element(s), sugar(s), antibiotic(s), antioxidant(s), etc. Typically, a medium supplement is a concentrated solution of the desired supplement to be diluted into a complete or base medium to reach the appropriate final concentration for cell culture.

As used herein, “serum replacement” or “serum replacement medium” refers to a composition that can be used in conjunction with a basal medium or as a complete medium in order to promote cell growth and survival in culture. Serum replacement is used in basal or complete medium as a replacement for any serum that is characteristically added to medium for culture of cells in vitro. It is contemplated that the serum replacement comprises proteins and other factors for growth and survival of cells in culture. The serum replacement is added to a basal medium prior to use in cell culture. It is further contemplated that a serum replacement may comprise a base medium and base nutrients such as salts, amino acids, vitamins, trace elements, antioxidants, and the like, such that the serum replacement is useful as a serum-free complete medium for cell culture.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Disclosed herein, are cell culture medium supplements that comprise at least one plant protein homologue of an animal protein and methods of making the same. In some embodiments, the animal protein is a serum protein. Also disclosed herein are methods of culturing cells in the disclosed cell culture medium supplements and utilizing said cultures for the production of cultured meat. The disclosed cell culture medium supplements disclosed herein can also be used in cell culture medium and kits. It was surprisingly discovered that plant protein homologues can be utilized in cell cultures in a manner that allows for the cell culture to be devoid of any animal proteins and/or animal components. The use of such plant-based proteins in culture media significantly reduces the cost of the medium for the production of cultured meat.

One aspect of the present disclosure provides a cell culture medium supplement comprising at least one plant protein homologue of an animal protein. The animal protein may be a serum protein. As such, in some embodiments, a cell culture medium supplement comprising at least one plant protein homologue of a serum protein is provided. The supplement may be devoid of any animal serum proteins. The supplement may also be essentially devoid of any animal serum-derived components.

The at least one plant protein homologue may be a homologue of a serum albumin, a serum catalase, a serum superoxide dismutase, a serum transferrin, a serum fibronectin, a serum vitronectin, a serum insulin, a serum hemoglobin, a serum aldolase, a serum lipase, a serum transaminase, a serum aminotransferase, a serum fetuin, or a combination thereof.

In some embodiments, the at least one plant protein homologue is a plant albumin, a plant catalase, a plant superoxide dismutase, a plant transferrin, a plant fibronectin, a plant vitronectin, a plant insulin, a plant leghemoglobin, a plant aldolase, a plant lipase, a plant transaminase, a plant aminotransferase, a plant cystatin, or a combination thereof.

In some embodiments, the at least one plant protein homologue comprise a plant albumin, a plant catalase, a plant fibronectin, and a plant insulin. In some embodiments, the at least one plant protein homologues further comprise a plant vitronectin. In some embodiments, the at least one plant protein homologues further comprise a plant superoxide dismutase. In some embodiments, the at least one plant protein homologues further comprise a plant transferrin.

The at least one plant protein homologue may be a plant albumin homologue. In some embodiments, the plant albumin homologue is from the water soluble fraction of a plant protein isolate. The water soluble fraction of a plant protein isolate may comprise plant albumins and globulins.

Albumin is a family of globular proteins, generally related to the globulin protein family. Albumins are water soluble proteins, moderately soluble in concentrated salt solutions, and experience heat denaturation.

Animal albumins are commonly found in blood plasma. Unlike other blood proteins, they are not glycosylated. Most commonly characterized and medically used albumin is Bovine Serum Albumin (BSA), 65-70 kilodalton (Kd). These serum albumins comprise of three homologous domains that assemble to form a heart-shaped protein. Each domain is a product of two subdomains that possess common structural motifs. Other albumin types include the storage protein ovalbumin in egg white, and different storage albumins in the seeds of some plants. Albumin binds to the cell surface receptor albondin, but can also enter the cell membrane through pinocytosis.

Serum albumins play a significant role in maintain the oncotic pressure of blood, and are utilizes as critical carrying proteins to deliver fatty acids, lipids, and growth factors to cells. In a preferred embodiment, an albumin homologue is a lipid carrier that can be used at a concentration sufficient to bind at least 50 μM oleic acid.

Plant albumins are abundant in seeds of many plants, such as pea (Croy et al., Biochem J. 1984 Mar. 15; 218(3): 795-803), lentils (Neves et al., Arch Latinoam Nutr. 1996 September; 46(3): 238-42) and hemp (Wang et al., 2019, Comprehensive Reviews in Food Science and Food Safety, 18(4): 936-952). They are also common in starchy plant roots like potatoes (Jirgensons, 1946, Journal of Polymer Science, 1(6): 484-494). Plant albumins are generally function as storage proteins. They are usually identified as 45-55 Kd homo dimers (Croy et al., Biochem J. 1984 Mar. 15; 218(3):795-803)

Plant storage albumins are broken-down during seed germination to provide nitrogen and sulfur for the developing seedling. During seed maturation these proteins are subject to post-translational modifications and trafficking before they are deposited in great quantity and with great stability in dedicated vacuoles (Mylne et al., 2014, Functional Plant Biology, 41(7): 671-677). Sharma et al. (Planta. 2015 May; 241(5): 1061-73) provides a crystal structure of a plant albumin from Cicer arietinum (chickpea) possessing hemopexin fold and hemagglutination activity. Dziuba et al. (Acta Sci. Pol., Technol. Aliment. 2014, 13(2): 181-190) provides proteomic analysis of albumin and globulin fractions of pea (Pisum Sativum L.) seeds.

Albumins were identified and purified in potatoes, pea, corn, soy, wheat, barley, rye, oat, and millet to replace bovine serum albumin in culture. In some embodiments, the plant albumin is a chickpea albumin, a hempseed albumin, a lentil albumin, a pea albumin, a soy albumin, a wheat albumin, a potato albumin, or combinations thereof. In another embodiment, the plant albumin is a pea albumin, a potato albumin, or combinations thereof. In an embodiment, the plant albumin is a pea albumin. In an embodiment, the plant albumin is a potato albumin.

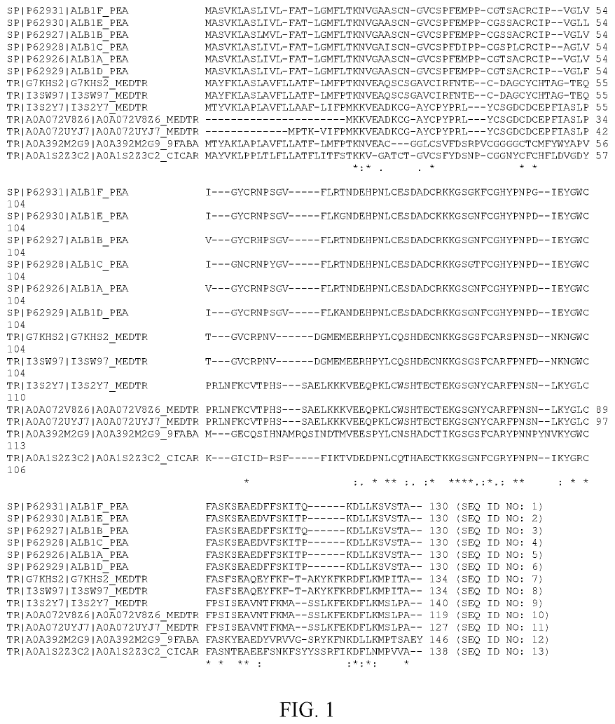

In some embodiments, the plant albumin comprises an albumin of a legume. Non-limiting examples include albumins of Pisum sativum (Garden pea) (UniProtKB: P62931, P62930, P62927, P62926, P62928, P62929), Medicago truncatula (Barrel medic) (UniProtKB: G7KHS2, I3SW97, I3S2Y7, A0A072V8Z6, A0A072UYJ7), Trifolium medium (UniProtKB: A0A392M2G9) and Cicer arietinum (Chickpea) (UniProtKB: A0A1S2Z3C2). Alignment of such proteins shows substantial sequence variation within a family of proteins that demonstrate a high degree of sequence similarity and function (e.g. nutrient storage activity). For reference, an alignment of the aforementioned proteins is depicted in FIG. 1, which shows a comparison of 13 legume albumins that are homologues of serum albumin. In detail, alignment of P62927 (Pea albumin) with A2U01_0001997 (Trifolium medium) over the sequence of P62927 from 71 to 113 compared with A2U01_0001997 from 78 to 122, 29 out of 43 amino acids are identical, and 34 out of 43 amino acids are identical or conserved. In non-conserved regions, homologues of the invention have more variation, for example, at least 95% identity, at least 90% identity, at least 85% identity, at least 80% identity, at least 70% identity, at least 60% identity, or at least 50% identity. The alignment exemplifies identification of families of plant proteins that are homologues of serum proteins by one of ordinary skill. As to mutations such as substitutions, insertions and deletions, the alignment exemplifies regions of high sequence identity and similarity.

In some embodiments, the plant albumin comprises a patatin or patatin homologue. Patatins comprise a family of glycoproteins and a major tuber storage protein. Patatins are found in potatoes and other nightshades such as capsicum, tobacco, and tomato. Patatins have been shown to have esterase activities including lipid acyl hydrolase (LAH) and acyl transferase activities. Non-limiting examples of plant albumins were identified in potato extracts by MS include patatins (UniProtKB: M1AGX5, Q2MYP6, Q2VBI2, Q2VBJ3, A0A097H149) and patatin-like phospholipase domain-containing proteins (PNPLAs) (UniProtKB: M1B3W0). Serum protein homologues comprise, without limitation, patatins and patatin fragments comprising the amino acid sequences set forth by the following UniProtKB accession numbers: M1AGX5, P15477, Q2MY51, Q2MY37, Q2MY45, Q2MY36, P11768, Q3YJT2, Q2MY52, P15476, Q2MY42, Q2MY41, P07745, Q8LPW4, Q2MY48, Q2MY40, Q2MY44, P15478, Q42502, Q3YJT3, Q3YJTO, Q2MY56, Q2MY58, Q2MY54, Q2MY43, Q2MY50, Q2MY60, Q2MY39, Q2MY38, Q3YJT5, Q2MY59, Q2MY55, Q41487, Q3YJT4, Q3YJS9, Q8LSC1, Q2VBI5, A0A1S3YWX7, A0A1J6HXU4, A0A2G3DDK1, A0A1U8EX11, A0A1U8EML3, A0A2G2W419, A0A1U8EMR5, A0A2G3DDL4, M1BFJ1, A0A0V0HSH3, A0A1J6KIW4, 024152, A0A1J6I2H9, A0A1U7XFQ0, and A0A1S4BJQ4.

In other embodiments, patatin fragments of any of the whole patatins correspond in size and location to the fragments set forth and may comprise the amino acid sequences set forth by the following UniProtKB accession numbers: Q9AUH5, Q2VB18, Q2VBJ4, Q9SB18, D1MI89, Q2VBI5, I6XCX7, Q2MYQ6, Q41475, Q2MYGO, Q2VBI9, Q7DMV4, Q2VBJ3, Q2VBI2, and Q2MYP6. As illustrative examples, the 51 amino acid (aa) homologue set forth in Q9AUH5 is a fragment of Q2MY48 from aa 92 to aa 142. The 132 aa fragment set forth in Q2VB19 is a fragment of Q2MY58 from aa 216 to aa 387. Similarly, the 132 aa fragment set forth in Q2VBJ4 is a fragment of Q2MY56 from aa 216 to aa 387. The 18 aa fragment set forth in Q9SB18 is a fragment of Q8LPW4 from aa 369 to aa 386. Fragments of any patatin corresponding in approximate size and location to the examples set forth comprise serum protein homologues of the invention.

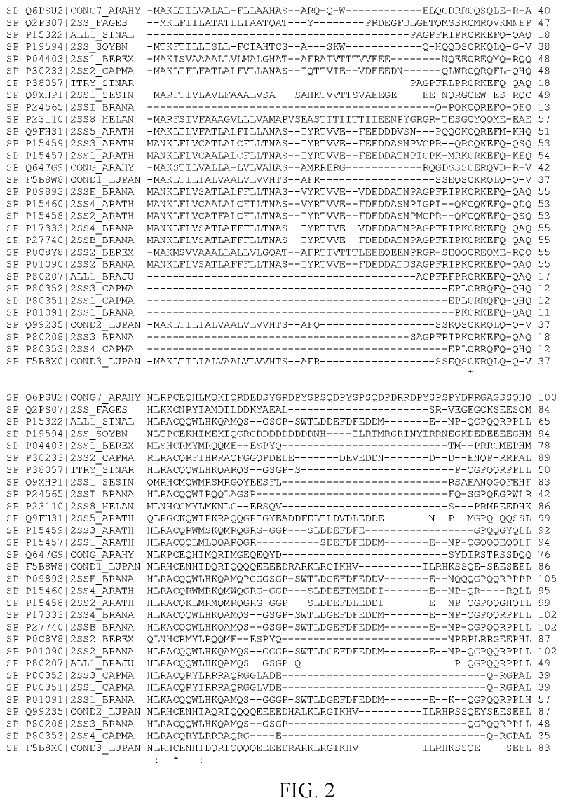

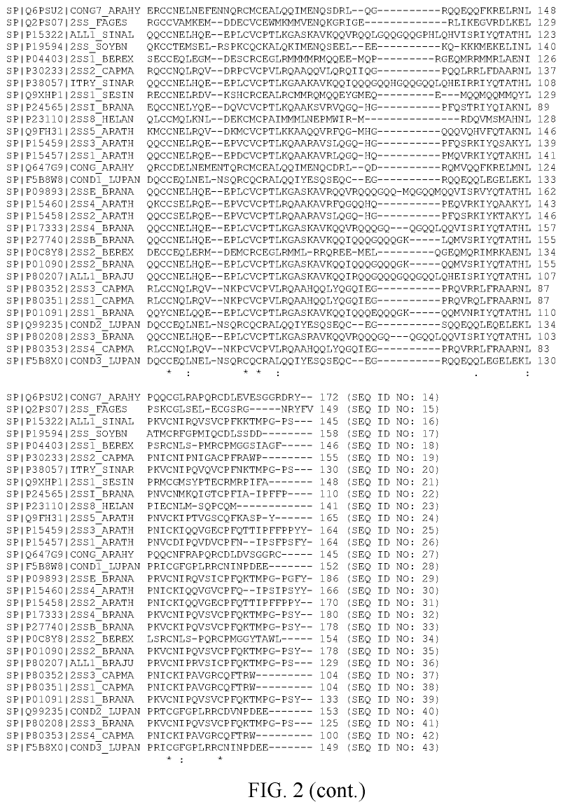

In some embodiments, the plant albumin comprises a seed storage albumin or albumin-like protein. Such proteins may comprise activities and functions such as nutrient reservoir, antimicrobial or anti-fungal, serine-type endopeptidase inhibitor, Non-limiting examples include Arachis hypogaea (Peanut) (UniProtKB: Q6PSU2, Q647G9), Fagopyrum esculentum (Common buckwheat) (Polygonum fagopyrum) (UniProtKB: Q2PS07), Ricinus communis (Castor bean) (UniProtKB: P01089, B3EWN4), Sinapis alba (White mustard) (Brassica hirta) (UniProtKB: P15322), Sinapis arvensis (Charlock mustard) (Brassica kaber) (UniProtKB: P38057), Brassica juncea (Indian mustard) (Sinapis juncea) (UniProtKB: P80207), Brassica rapa subsp. chinensis (Pak-choi) (Brassica chinensis) (UniProtKB: P84529), Glycine max (Soybean) (Glycine hispida) (UniProtKB: P19594), Hemp (UniProtKB: A0A219D1L6) Bertholletia excelsa (Brazil nut) (UniProtKB: P04403, POC8Y8), Capparis masaikai (Mabinlang) (UniProtKB: P30233, P80352, P80351, P80353), Sesamum indicum (Oriental sesame) (Sesamum orientale) (UniProtKB: Q9XHP1, B3EWE9), Taraxacum officinale (Common dandelion) (Leontodon taraxacum) (UniProtKB: P86783), Brassica napus (Rape) (UniProtKB: P24565, P09893, P17333, P27740, POC8Y8, P80208), Cucurbita maxima (Pumpkin) (Winter squash) (UniProtKB: Q39649), Helianthus annuus (Common sunflower) (UniProtKB: P23110, P15461), Arabidopsis thaliana (Mouse-ear cress) (UniProtKB: Q9FH31, P15457, P15460, P15458), Lupinus angustifolius (Narrow-leaved blue lupine) (UniProtKB: F5B8W8, Q99235, F5B8X0, F5B8X1), Oryza sativa subsp. japonica (Rice) (UniProtKB: P29835), Matteuccia struthiopteris (European ostrich fern) (Osmunda struthiopteris) (UniProtKB: P17718), Cucurbita moschata (Winter crookneck squash) (Cucurbita pepo var. moschata) (UniProtKB: P84576), Passiflora edulis (Passion fruit) (UniProtKB: P84884), and Picea glauca (White spruce) (Pinus glauca) (UniProtKB: P26986). Alignment of such proteins shows substantial sequence variation but within a family of proteins that demonstrate a high degree of sequence similarity and function. For reference, an alignment of a selection of the aforementioned proteins is depicted in FIG. 2, which shows a comparison of 30 seed storage albumin homologues of serum albumin. The alignment exemplifies identification of families of plant proteins that are homologues of serum proteins. As to mutations such as substitutions, insertions and deletions, the alignment shows regions of high sequence identity and similarity.

The plant albumins generally break down to high molecular-weight albumins of ˜50-110 Kd, average molecular-weight albumins of ˜20-35 Kd, and low molecular-weight albumins of ˜13-17 Kd. Plant albumins usually function as homodimers. In some embodiments, the plant albumin has a molecular weight of about 13-110 kilodaltons (Kd). In some embodiments, the plant albumin has a molecular weight of about 13-17 Kd. In some embodiments, the plant albumin has a molecular weight of about 20-35 Kd. In some embodiments, the plant albumin has a molecular weight of about 50-110 Kd.

The plant albumin may have a concentration of about 0.01% to about 10% (w/w) in the medium. In some embodiments, the plant albumin may have a concentration of about 0.01% to about 5% (w/w) in the medium. In some embodiments, the plant albumin is at a concentration of about 0.01% to about 5% by weight in the cell culture medium. In some embodiments, the plant albumin is at a concentration of about 0.01% to about 0.05% by weight, about 0.01% to about 0.05% by weight, about 0.05% to about 0.1% by weight, about 0.1% to about 0.15% by weight, about 0.15% to about 0.2% by weight, about 0.2% to about 0.25% by weight, about 0.25% to about 0.3% by weight, about 0.3% to about 0.35% by weight, about 0.35% to about 0.4% by weight, about 0.4% to about 0.45% by weight, or about 0.45% to about 0.5% by weight.

It will be appreciated by those of skill that the amount of a given component in a cell culture medium supplement as disclosed herein, can be calculated so as to provide the desired concentration in the final weight or volume of the cell culture medium. For example, one of skill can determine the appropriate concentration of plant albumin to include in a cell culture medium supplement, such that when the cell culture medium supplement is added to a cell culture medium the final concentration of the plant albumin in the cell culture medium is as desired (e.g., 0.01% to about 5% w/w in the final cell culture medium).

The at least one plant protein homologue may be a plant catalase.

Excessive hydrogen peroxide is harmful for almost all cell components, so its rapid and efficient removal is of essential importance for aerobically living organisms. Cells grow and thrive in medium supplemented with FBS. It has been long known that FBS has a protective role in cell culture, and its removal decreases viability of cells immensely. Lieberman and Ove (J Exp Med. 1958 Nov. 1;108(5): 631-7), have shown that this decrease in viability could be rescued using protein extracts from liver, and the enzyme catalase was then identified as the key component there. Therefore, defined supplements that support serum free culture of cells, including a B27 supplement (aka B22 supplement from the Hanna lab) contain high amounts of catalase.

Catalase in cell culture is most often derived from human erythrocytes (e.g. Sigma #C3556) or bovine liver (e.g. Sigma #C1345). Catalase can also be produced from bacteria such as Micrococcus lysodeikticus (e.g. Sigma #60634) or Corynebacterium glutamicum (e.g. Sigma #02071) or from fungi such as Aspergillus niger (e.g. Sigma #C3513).

Catalase (CAT) is a common antioxidant enzyme in almost all living organisms. It catalyzes the breakdown of hydrogen peroxide to water and oxygen, and thus protects the cell from oxidative damage by reactive oxygen species (ROS). Catalase function is evolutionarily conserved from bacteria to humans (Zamocy et al., Antioxid Redox Signal, 2008 September; 10(9): 1527-1548). Plant Catalase family comprises 3 catalases, which are expressed abundantly in leaves and roots (Sharma and Ahmad, Chapter 4—Catalase: A Versatile Antioxidant in Plants Oxidative Damage to Plants Antioxidant Networks and Signaling, 2014, P. 131-148). Active catalase can be isolated from plants quite easily, and its protective function can be tested in tissue culture, using a defined supplement for serum free culture in which the bovine liver catalase is exchanged with potato/cabbage catalase (Gholamhoseinian et al., 2006, Asian Journal of Plant Sciences, 5(5): 827-831) or cucumber catalase (Hu et al., Genetics and Molecular Biology, 39(3): 408-415, 2016).

Catalase is a common water soluble enzyme found in nearly all living organisms exposed to oxygen. It catalyzes the decomposition of hydrogen peroxide to water and oxygen. It helps protect the cell from oxidative damage by reactive oxygen species (ROS). Its turnover rate is one of the highest known in nature.

Human catalase is a tetramer of four polypeptide chains, each over 500 amino acids long. It contains four iron-containing heme groups that allow the enzyme to react with the hydrogen peroxide.

Plant catalases differ in their optimal temperature and pH range, depending on their growth conditions. They are most notably distinguished from other enzymes that can metabolize peroxidesin by not requiring a reductant as they catalyse a dismutation reaction (Mhamdi et al., 2010, Journal of Experimental Botany, 61(15): 4197-4220). These enzymes consist of polypeptides of 50-70 Kd in mass that are organized into tetramers, with each monomer bearing a haem prosthetic group (Regelsberger et al., Plant Physiology and Biochemistry, 2002, 40: 479-490).

A second type of haem-dependent catalase is bifunctional catalase-peroxidases that are structurally distinct proteins found in some fungi and prokaryotes (Mutsada et al., Biochemical Journal, 1996, 316: 251-257; Regelsberger et al., Plant Physiology and Biochemistry, 2002, 40: 479-490).

According to Martins and English (RedoxBiology, 2014, 2: 308-313), catalase activity is stimulated by H₂O₂ in rich culture medium and is required for H₂O₂ resistance and adaptation in yeast.

Multiple molecular forms of catalase have been reported in different plant species, e.g. Nicotiana tobacco (Havir and McHale, 1987, Plant Physiol. 84: 450-455), cotton (Ni et al., 1990, Biochim. Biophys. Acta. 1049: 219-222), Nicotiana plumbaginlfolia (Willekens et al., 1994, FEBS Lett. 352: 79-83), Arabidopsis thaliana (Zhong et al., 1994, Plant Physiol. 104: 889-898), Pinus taeda (Mullen and Gifford, 1993, Plant Physiol. 103: 477-483), sunflower (Eising et al., 1989, Arch. Biochem. Biophys. 278: 258-264), pumpkin (Yamaguchi et al., 1986, Eur. J Biochem. 159: 315-322) and tomato (Gianinetti et al., 1993, Physiol. Plant. 89: 157-164). Catalase nomenclature is also based on its isoforms in different plant species. According to a classification suggested by Willekens et al. (1995, EMBO J. 16: 4806-4816), Class I, Class II and Class III catalases are specifically expressed in photosynthetic tissues, vascular tissues and reproductive tissues, respectively. The presence of multiple catalase isozymes suggests structural and functional versatility of catalases in a variety of plant species. The cDNA of various catalases has been isolated and characterized from different plant species to understand genes and their regulatory components (Scandalios, 1992, Current Communications in Cell and Molecular Biology: Molecular Biology of Free Radical Scavenging Systems (5). Cold Spring Harbor Laboratory Press, Cold Spring Harbor, N.Y.). The isozymes of catalase exhibit developmental stage and organ specificity in plants.

The plant catalase may be an Arabidopsis catalase, a cabbage catalase, a cucumber catalase, a cotton catalase, a potato catalase, a pumpkin catalase, a spinach catalase, a sunflower catalase, a tobacco catalase, a tomato catalase, or combinations thereof. In an embodiment, the plant catalase is a cabbage catalase, a cucumber catalase, a potato catalase, or combinations thereof. In an embodiment, the plant catalase is a cucumber catalase. In an embodiment, the plant catalase is a potato catalase.

The plant catalase may have a molecular weight of about 50-70 kilodaltons.

In some embodiments, the concentration of plant catalase in the medium may be at a concentration of about 1 to about 100 ng/ml, e.g., about 7 ng/ml, about 11 ng/ml, about 14 ng/ml, about 18 ng/ml, about 21 ng/ml, about 28 ng/ml, about 35 ng/ml, or about 55 ng/ml. In some embodiments, the final concentration of plant catalase in the medium may be about 1 g/l to about 5 g/l and advantageously at 2.5 g/1.

The UniProt database contains at least 128 proteins that have 90% identity to potato catalase (UniProtKB: M1ALT0) and at least 958 proteins that have 50% identity to potato catalase. Similarly, the UniProt database contains at least 23 proteins that have 90% identity to wheat (Triticum) catalase (UniProtKB: Q43206) and at least 958 proteins that have 50% identity to wheat catalase. Sources of plant catalases used according to the invention and exemplary catalases from those plants further include without limitation, soybean (UniProtKB: 048561), chickpea (UniProtKB: A0A1S2Y835, Q9ZRU4), Cucurbita pepo (Summer squash) (UniProtKB: P48350), mung bean (UniProtKB: P32290), kidney bean (UinProtKB: T2DN96, V7AQS4), and cotton (UniProtKB: P17598, A0A5D2M8G9, A0A5J5SMB2). Another viridiplantae database resource is Phytozome, the Plant Comparative Genomics portal of the Department of Energy's Joint Genome Institute (Heinze, et al., (2002) Plant Catalases. In: Baker A., Graham I. A. (eds) Plant Peroxisomes. Springer, Dordrecht).

As described and exemplified herein, plant homologues of serum catalase and regions of conserved and non-conserved sequences among proteins of the families and between individual members are readily recognized. As to mutations such as substitutions, insertions and deletions, the alignments locate the regions of highest sequence identity and similarity.

Isolation of plant catalases is well known to one of skill in the art and involves liquid fractionation, centrifugation, exchange columns, all of which is routine experimentation. Catalase was identified in potatoes in the present disclosure.

The at least one plant protein homologue may be a plant fibronectin, a plant vitronectin, or combinations thereof.

Cell survival in culture is often dependent the surface provided for attachments. In vivo, the extracellular matrix (ECM) proteins of the basement membrane allow cells to adhere to neighboring tissues by engaging the integrin family of surface glycoproteins. Laminin, collagen IV and heparan sulfate constitute the basement membrane proteins in adult tissues, with embryonic and regenerating tissues also showing fibronectin. Many of the same ECM proteins are derived from animals or expressed recombinantly to support cell attachment and growth in vitro.

In plants, the role of the ECM like proteins in support and anchorage has also been recognized. Several animal ECM like proteins were discovered over the years in the plant cell wall (Seymorr et al. 2004, Biotechnology and Genetic Engineering Reviews, 21(1): 123-132). Fibronectin-like protein have been shown to be involved in cell wall-plasma membrane attachment and are enriched under conditions of salt stress/water deficit (Zhu et al., 1993). Pellenc and colleagues (Pellenc et al., 2004, Protein Expression and Purification 34: 208-214) were able to isolate a fibronectin like protein from plants, using a protocol similar to that of fibronectin isolation from human plasma.

Tobacco proteins immunologically related to human vitronectin are found in cell walls and membranes of unadapted and salt adapted tobacco cells, enriched in the adapted cells. (Zhu, J. K. et al., Plant J. for Cell and Molecular Biology, 30 Apr. 1993, 3(5): 637-646). Sanders discovered antibodies specific for the 55 Kd polypeptide of tobacco cells were also able to recognize human Vn (Sanders et al., 1991, Plant Cell, 3: 629-635). Mono-specific antibodies specific for the 59 Kd protein of tobacco microsomal membranes, recognize human fibronectin.

The plant homologue of a fibronectin may be a bean fibronectin (UniProtKB: V7C3U9, V7CSV1, A0A0L9VRR4, A0A1S3UT35, A0A1S3UV51) chickpea fibronectin (UniProtKB: A0A1S2Z0R0, A0A1S2YDZ6, A0A1S3E9P2, A0A1S3E9K8, A0A1S2YE00), a lentil fibronectin, a rice fibronectin (UniProtKB: 1NXC4, A0A0E0JVM6, J3L9M7, A0A0E0N9Z2), a soy fibronectin (UniProtKB: I1MSQ1, I1KIT9, K7L4U6, A0A0R0IKE0) a tobacco fibronectin (UniProtKB: A0A1J6IU80, A0A1S4B3E7, A0A1S4AF52, A0A1J6HY83) or a wheat fibronectin (UniProtKB: A0A3B6NN97, A0A3B6KF25, A0A3B6QBJ6), or a chickpea blight fungus fibronectin (UniProtKB: A0A163LS C5, A0A163GQI0).

Fibronectin and vitronectin like proteins were identified in several plants, and crude plant extracts were used to support the attachment and growth of cells in the cell culture media.

In some embodiments, the at least one plant protein homologue may be a plant fibronectin. In some embodiments, the plant fibronectin is a bean fibronectin, a chickpea fibronectin, a lentil fibronectin, a rice fibronectin, a soy fibronectin, a tobacco fibronectin, a wheat fibronectin or combinations thereof. In some embodiments, the plant fibronectin is a chickpea fibronectin, a lentil fibronectin, a rice fibronectin, a soy fibronectin, a wheat fibronectin, or combinations thereof. In an embodiment, the plant fibronectin is a lentil fibronectin. In an embodiment, the plant fibronectin is a rice fibronectin. In an embodiment, the plant fibronectin is a soy fibronectin. In an embodiment, the plant fibronectin is a wheat fibronectin.

The plant fibronectin may have a molecular weight of about 40-60 Kd. Animal serum fibronectins are often larger, consisting of two subunits of about 250 Kd. In some embodiments, the plant fibronectin has a molecular weight of about 40-60 Kd.

In some embodiments, the plant fibronectin is at a concentration of about 0.1 μg/ml to about 100 μg/ml in the medium. In some embodiments, the plant fibronectin is at a concentration of about 1 μg/ml, about 2 μg/ml, about 3 μg/ml, about 4 μg/ml, μg/ml, about 5 μg/ml, about 6 μg/ml, about 7 μg/ml, about 8 μg/ml, about 9 μg/ml, about 10 μg/ml, about 15 μg/ml, about 20 μg/ml, about 30 μg/ml, about 40 μg/ml or about 50 μg/ml in the medium.

In some embodiments, the at least one plant protein homologue may be a plant vitronectin.

In some embodiments, the plant vitronectin is at a concentration of about 0.1 μg/ml to about 100 μg/ml in the medium. In some embodiments, the plant vitronectin is at a concentration of about 1 μg/ml, about 2 μg/ml, about 3 μg/ml, about 4 μg/ml, μg/ml, about 5 μg/ml, about 6 μg/ml, about 7 μg/ml, about 8 μg/ml, about 9 μg/ml, about 10 μg/ml, about 15 μg/ml, about 20 μg/ml, about 30 μg/ml, about 40 μg/ml or about 50 μg/ml in the medium.

Isolation of plant fibronectin and vitronectin is well known to one of skill in the art and involves liquid fractionation, centrifugation, exchange columns, all of which is routine experimentation.

The at least one plant protein homologue may be a plant leghemoglobin.

In some embodiments, the plant leghemoglobin is at a concentration of about 1 μg/ml to about 100 μg/ml in the medium.

Leghemoglobins are oxygen carrier proteins found in the nitrogen-fixing root nodules of leguminous plants, produced by legumes in response to the roots being colonized by nitrogen-fixing bacteria. The leghemoglobins comprise serum-protein homologues of the invention. Leghemoglobins include, without limitation, Pisum sativum (Garden pea) (UniProtKB: LGB1_PEA, LGB2 PEA, LGB3_PEA, LGB4_PEA, LGB5_PEA, LGB6 PEA), Medicago sativa (Alfalfa) (UniProtKB: LGB1_MEDSA, LGB2_MEDSA, LGB4_MEDSA, Q42928_MEDSA, Q43786 MEDSA, Canavalia lineata (Beach bean) (Dolichos lineatus) (UniProtKB: LGB_CANLI), Cicer arietinum (Chickpea) (Garbanzo)(UniProtKB: A0A1S2YZ78, A0A1S2XXT5, A0A1 S2XKV 1, A0A1S2XMF3), Glycine max (Soybean) (Glycine hispida) (UniProtKB: A0A0R0HW51, Q96428, Q42801), Glycine soja (Wild soybean)(UniProtKB: A0A445IPX6), and Medicago sativa (Alfalfa) (UniProtKB: P28010, 14962, Q42928, Q43786)

The at least one plant protein homologue may be a plant lipase.

In some embodiments, the plant lipase is at a concentration of about 1 μg/ml to about 100 μg/ml in the medium.

Additional non-limiting examples of serum protein homologues include the following homologues of serum proteins. Lipase is an enzyme that catalyzes breakdown of fats to fatty acids and glycerol or other alcohols. A multitude of lipases can be found among plants from sources including garden pea (Pisum sativum) (UniProtKB: Q01517), Triticum aestivum (wheat) (UniProtKB: A0A1D5UIX7, A0A2X0SGN9, A0A3B6GZV3, A0A3B6GY43) Arabidopsis thaliana (Mouse-ear cress) (UniProtKB: A0A178WBX6) and many others. Plant lipase preparations methods are well-known in the art (see, e.g. Wagenknecht, A. C. et al., 1958, Journal of Food Science, 23(5): 439-445; Barros, M. et al., 2010, Brazilian Journal of Chemical Engineering 27(1): 15-29).

The at least one plant protein homologue may be a plant cystatin.

In some embodiments, the plant cystatin is at a concentration of about 1 μg/ml to about 100 μg/ml in the medium.

Fetuins are blood proteins that are made in the liver and secreted into the bloodstream that includes serum albumin. They belong to a group of binding proteins mediating the transport and availability of a wide variety of cargo substances in the bloodstream. Whereas serum albumin is the most abundant protein in the blood plasma of adult animals, fetuin is more abundant in fetal blood. Fetuin-A is a major carrier protein of free fatty acids in the circulation. Fetuin-A has been reported to play a role in cellular adhesion and signaling, and to modulate growth, motility, and invasion of certain cancer cell types. Fetuins belong to the cystatin superfamily of proteins and evolved from the protein cystatin by gene duplication and exchange of gene segments. In mammals, fetuin-A and fetuin-B are paralogous plasma proteins of the cystatin superfamily (see, e.g., Karmilin et al., 2019, Sci Rep. 2019; 9: 546). Many cystatins have been identified as inhibitors of papain-like cysteine proteinases. While fetuin-A is not known to be an inhibitor of a protease, fetuin-B selectively inhibits certain metalloproteinases. Mammalian cystatins generally are cysteine-protease inhibitors and found in all biological fluids. Mammalian cystatin C is a secreted protein, can be internalized by cells (Ekström, U. et al., 2008, FEBS Journal 275: 4571-4582) and is used in cell culture applications where it can inhibit intracellular processes, including inhibiting polio, herpes simplex and coronavirus replication.

The MEROPS database classifies cystatin proteins as members of the 125 family. The cystatin family (designated 125) comprises cysteine protease inhibitors including cystatins classified in subdivided into four subfamilies: I25A, I25B, I25C, and unclassified. (Rawlings et al., 2014, Nucleic Acids Res. 42: D503-D509; Martinez, M. et al., 2009, Plant Physiol. 2009, November; 151(3): 1531-45). Plant cystatins (classified as phytocystatins) comprise an N-terminal alpha-helix (present only in plant cystatins) and have mainly been identified from seeds, and some have been detected in other plant tissues. Multicystatins of potato (Solanum tuberosum) and tomato (Solanum lycopersicum) can be found in vacuoles and in cytoplasm. (Nissen et al., 2009, Plant Cell 21: 861-875; Madureira et al, 2006, Environ Exp Bot 55: 201-208).

Plant homologues of serum proteins include plant cystatins. One is the presence of a N-terminal alpha-helix, present only in plant cystatins. Non-limiting examples include a Vigna unguiculata (cowpea) cystatin (UniProtKB: A0A4D6KLC0, A0A4D6NH52), a Glycine max (Soybean) cystatin (UniProtKB: I1K3Q1, P25973, A0A0R4J598, IlMYC1), a Hordeum vulgare (Barley) cystatin (UniProtKB: Q9LEI7), a Oryza sativa (rice) cystatin (UniProtKB: A2XS65, Q6K309, A0A1S4AF52, A0A1J6HY83), a Solanum tuberosum (potato) cystatin (UniProtKB: P37842, M1B0W4, M10699, M1B0W5, M1BIR8), a Zea mays (maize) cystatin (UniProtKB: P31726, B6SNY0, B6UGN8), a Triticum (wheat) cystatin (UniProtKB: Q8W252), a Phaseolus vulgaris (kidney bean) cystatin (UniProtKB: V7C6Q5, V7BNT8), a Arachis hypogaea (peanut) cystatin (UniProtKB: A0A445AB69, A0A445DKL4, E5BDA5), a Helianthus annuus (common sunflower) cystatin (UniProtKB: Q10992, Q109923), or a Dictyostelium discoideum (slime mold) cystatin (UniProtKB: Q65YR7, Q65YR8, Q5R1U3) (see the viridiplantae database resource Phytozome and UniProt for cystatins).

The at least one plant protein homologue may be a plant aldolase.

In some embodiments, the plant aldolase is at a concentration of about 1 μg/ml to about 100 μg/ml in the medium.

Aldolase is an enzyme that helps break down certain sugars, is found in high amount in muscle tissue, and is detectable in blood. An example of a plant homologue is Fructose-1,6-bisphosphate aldolase (FBA), a key plant enzyme that is involved in glycolysis, gluconeogenesis, and the Calvin cycle (Lv et al., 2017, Front Plant Sci. 8:1030). Another source is garden pea (UniProtKB: Q01517).

Other suitable plant homologues of proteins include transaminases and aspartate aminotransferases.

The at least one plant protein homologue may be a plant transaminase. Non-limiting examples of transaminases include garden Pea (Pisum sativum) (UniProtKB: P49364, Q9AVH0, 022464) and Triticum (Wheat) (UniProtKB: P84188). In some embodiments, the plant transaminase is at a concentration of about 1 μg/ml to about 100 μg/ml in the medium.

The at least one plant protein homologue may be a plant aminotransferase. Matheron describes purification and properties of an aminotransferase of garden pea (Matheron et al., Plant Physiol. 1973, 52: 63-67). Non-limiting examples of aspartate aminotransaminases include Soy (Glycine max) (UniProtKB: I1JUS6) and Triticum (Wheat) (UniProtKB: B5B1F8). In some embodiments, the plant aspartate aminotransferase is at a concentration of about 1 μg/ml to about 100 μg/ml in the medium.

Insulin homologues from plant sources can also be used as a cell culture supplement as part of the disclosure.

In some embodiments, structural or functional homologues of insulin may be included in the cell culture medium supplement. Examples of such insulin homologues include, but are not limited to, glucokinin, charantin, and corosolic acid. Glucokinin is a structural homologue of insulin. Charantin is a mixture of two steroid glycosides that is derived from Momordica charantia plant, or Bitter lemon. Corosolic acid is a pentacyclic triterpene acid found in Lagerstroemia speciose plant and is usually extracted from banana leaves.

In some embodiments, the plant insulin is glucokinin, charantin, corosolic acid, or combinations thereof. In an embodiment, the plant insulin is glucokinin. In an embodiment, the plant insulin is charantin. In an embodiment, the plant insulin is corosolic acid.

In some embodiments, the plant insulin has a molecular weight of about 6 Kd. In some embodiments, the plant insulin is at a concentration of about 0.05 μg/ml to about 10 μg/ml in the medium.

The at least one plant protein homologue may be a plant superoxide dismutase (SOD). In some embodiments, the plant SOD has a molecular weight of about 80-89 Kd. In some embodiments, the plant SOD is at a concentration of about 1 μg/ml to about 20 μg/ml in the medium.

The at least one plant protein homologue may be a plant transferrin.

Serum-replacement proteins specifically identified herein are exemplary and non-limiting. Orthologues and/or paralogues of the non-animal proteins are also included. Orthologues are often defined as homologous genes or proteins that are the result of a speciation event. In a simple model, following a speciation event, orthologues result when the gene or protein of the first and second species diverge. While the sequences of orthologues may differ, the orthologous proteins and encoding nucleotides tend to have the same or similar function or activity or fulfill the same role in different species, having been maintained through a speciation event. Paralogues are often defined as homologous genes or proteins that are the result of a duplication event in a species. In a simple model, paralogues result following a gene duplication event, and divergence of one copy from the other. Paralogues can evolve separately in the same species, thus tend to be more divergent in their roles, although their functions may be similar. For example, paralogues may have similar enzymatic activity but act on different substrates, or be expressed in different tissues, or at different stages of development. The relationship among orthologues and paralogues can be more complex, e.g. where there is a gene duplication followed by speciation.

Alternative embodiments include serum protein homologues comprising mutations, including substitutions, insertions, and deletions. Particular amino acid sequence variants may differ from a reference sequence by insertion, addition, substitution or deletion of 1 amino acid, 2, 3, 4, 5-10, 10-20 or 20-30 amino acids. In some embodiments, an alternative embodiment sequence may comprise the reference sequence with 1, 2, 3, 4, 5, 6, 7, 8, 9, 10 or more residues inserted, deleted or substituted. For example, 5, 10, 15, up to 20, up to 30 or up to 40 residues may be inserted, deleted or substituted.

For any particular plant homologue of a serum protein, comparison of non-animal homologues, including orthologues and paralogues, serves as a guide to mutations that can be made or selected. For example, it is evident from such sequence alignments which portions of the homologous proteins are more or less conserved and which portions may comprise greater or lesser variation. Thus, one way to determine whether a protein is a suitable non-animal serum protein homologue is to align homologues provided herein to identify which portions of the homologues comprise greater or lesser conservation or greater or lesser variation, or where there can be insertions and deletions, and then compare the protein in question with one or more of the homologues provided herein. A local alignment algorithm such as Smith and Waterman can be used to align two or more of the homologues. Such algorithm may be implemented on a computer to optimize the alignment. Several computer programs are available that employ the Smith and Waterman algorithm. For example, BestFit uses the Smith-Waterman algorithm to find the best local alignment between two sequences. Other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448).

Through alignments of multiple sequences, amino acid residues that are highly conserved or invariant are identified. The sequence comparisons highlight amino acid residues that are identical or nearly identical among the sequences and are likely to be important for function, amino acids that are conserved or highly conserved, and amino acids that are variable. The alignments also indicate where there are insertions or deletions from one protein to another, thus sequences of the proteins that can be dispensable.

Taking any one of the particularly disclosed proteins as a reference, homologues of the invention, including orthologues, paralogues and mutants thereof may differ from the reference in a conserved region by 1, 2, 3, 4, 5, 6, 7, 8, 9, 10 or more conservative substitutions. On can look to the pairwise comparisons of the disclosed sequences as a guide. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue.

Amino acids may be grouped into different classes according to common side-chain properties: a. hydrophobic: Met, Ala, Val, Leu, Ile; b. neutral hydrophilic: Cys, Ser, Thr, Asn, Gln; c. acidic: Asp, Glu; d. basic: His, Lys, Arg; e. residues that influence chain orientation: Gly, Pro; aromatic: Trp, Tyr, Phe. Non-conservative substitutions will entail exchanging a member of one of these classes for another class. Amino acids may be grouped into different classes according to common side-chain properties: a. hydrophobic: Met, Ala, Val, Leu, Ile; b. neutral hydrophilic: Cys, Ser, Thr, Asn, Gln; c. acidic: Asp, Glu; d. basic: His, Lys, Arg; e. residues that influence chain orientation: Gly, Pro; aomatic: Trp, Tyr, Phe. Non-conservative substitutions will entail exchanging a member of one of these classes for another class.

Conservative substitutions are shown in Table 1 below:

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

In some embodiments, homologues having at least 50%, at least 55%, at least 60%, at least 65%, at least 70%, at least 75%, at least 80%, at least at least 85%, at least 87%, at least 90%, at least 91%, at least 92%, at least 93%, at least 94%, at least 95%, at least 96%, at least 97%, at least 98%, or at least 99% sequence identity to an identified homologue are included.

In some embodiments, the homologues have at least 50% sequence identity to an identified homologue.

In some embodiments, the homologues have at least 60% sequence identity to an identified homologue.

In some embodiments, the homologues have at least 70% sequence identity to an identified homologue.

In some embodiments, the homologues have at least 80% sequence identity to an identified homologue.

In some embodiments, the homologues have at least 90% sequence identity to an identified homologue.

In some embodiments, the homologues have at least 95% sequence identity to an identified homologue.

Some embodiments include fusions of serum protein homologues to other proteins and polypeptides. The fusion proteins may display enhancements in production, activity, stability, and/or targeting. Serum protein analogs can be evaluated alone or in combination.

In some embodiments, the plant protein homologues may be glycoengineered. Glycosylation is one of the major post-translation protein modifications. N-linked glycosylation is the attachment of an oligosaccharide, a carbohydrate consisting of several sugar molecules, sometimes also referred to as glycan, to a nitrogen atom (the amide nitrogen of an asparagine (Asn) residue of a protein). O-linked glycosylation is the attachment of a sugar molecule to the oxygen atom of serine (Ser) or threonine (Thr) residues in a protein. Glycosylation is often essential for protein structure and function. N- and O-glycans have been shown to play important roles in protein structure, stability, aggregation, and thermal denaturation, and have been observed to influence pharmacodynamics and pharmacokinetics of recombinant therapeutic proteins. N- and O-linked carbohydrate moieties of plant and insect glycoproteins are also abundant environmental immune determinants.

Glycoengineering refers to selecting or remodeling of glycans. Glycoengineering involves selecting a host organism for expression. Non-human mammalian cells such as CHO have been used predominantly for the production of biopharmaceuticals having a human-like glycosylation profile.

Yeast and other fungal hosts are important production platforms for production of recombinant proteins. Cell lines of the yeast strain Pichia pastoris have been developed that carry out a sequence of enzymatic reactions which mimic the process of glycosylation in humans. For example, U.S. Pat. Nos. 7,029,872, 7,326,681, and 7,449,308 describe methods for producing a recombinant glycoprotein that is similar to a human protein, comprising sialylated bi-antennary complex N-linked glycans. Glycoengineering may involve expressing a protein in an organism engineered to do specific glycosylation.

Glycoengineering can be enzymatic, e.g., employing enzymes such as endoglycosidases and glycosynthases. Exemplary endoglycosidases include, but are not limited to, Endo-β-N-acetylglucosaminidase H (Endo-H) is a recombinant glycosidase which cleaves within the chitobiose core of high mannose and some hybrid oligosaccharides from N-linked glycoproteins. Endo-N-acetylglucosaminidase F2 (Endo-F2) cleaves high mannose and biantennary N-glycans and Endo-N-acetylglucosaminidase F3 (Endo-F3) cleaves triantennarry and alpha-(1-6)-fucosylated biantennary N-glycans from peptides and protein (Plummer et al., Anal Biochem 235: 98-101, 1996). Such enzymes can be used to digest an oligosaccharide to a single sugar unit (e.g., GlcNAc) which can then be elongated with an oligosaccharide of choice by glycosylation mediated by a glycosyltransferase. An α-fucosidase can be employed to de-fucosylate the asparagine-linked terminal GlcNAc. Glycosyltransferases for elongating the single sugar unit include, without limitation, endo-β-1,4-galactosyltransferase. Oligosaccharides may be sialylated. A sialyltransferase can be used to catalyze transfer of a sialic acid moiety to the terminal portions of an oligosaccharide acceptor. Each sialyltransferase is specific for a particular sugar substrate.

To completely remove an oligosaccharide, PNGase F, which is an amidase, cleaves between the innermost GlcNAc and asparagine residues of high mannose, hybrid, and complex oligosaccharides and is effective to remove almost all N-linked oligosaccharides and leaves the N-glycan core oligosaccharides intact for further analysis (WO 2013/120066).

Metabolic glycoengineering (MGE) is a technique for manipulating cellular metabolism to modulate glycosylation. MGE can be used to increase the levels of natural glycans as well as to substitute non-natural monosaccharides into glycoconjugates (Agatemor et al., Nat Rev Chem 3: 605-620 (2019)). For example, using MGE can be employed to feed metabolic substrates (e.g., ManNAc, Neu5Ac, and CMP-Neu5Ac analogs) into the sialic acid biosynthetic pathway resulting in non-natural sialoside display (Du et al., 2009, Glycobiology 19(12): 1382-401).

Certain non-mammalian (e.g., plant) proteins are not homologues of serum proteins of the present disclosure. For example, while a cell culture system may include components that comprise serine protease inhibition activity, a soy trypsin inhibitor is not the at least one plant protein homologue. Likewise, soy based antioxidants are not serum proteins homologues. Thus, in some embodiments the at least one plant protein homologue is not a trypsin inhibitor. In some embodiments, the at least one plant protein homologue is not a soy based antioxidants.

The plant protein homologues may be obtained from a number of sources.

In some embodiments, the plant protein homologue may be any plant extract or fraction of a plant extract that comprises at least one plant protein homologue of a serum protein. By way of a non-limiting example, the at least one plant protein homologue may be from or in the water soluble fraction of a plant protein extract. The water soluble fraction of the plant protein isolate may comprise plant albumins and plant globulins. In other embodiments, the water soluble fraction of the plant protein isolate may comprise plant albumin. Methods of obtaining plant extracts and fractions are known in the art.

The plant protein extracts may comprise one or more plant protein homologues. For example, in some embodiments, the plant protein isolate may comprise plant albumin and plant globulins. In other embodiments, the plant protein isolate may comprise plant albumin.

In some embodiments, the plant protein homologues are isolated from plant extracts. Isolation of plant proteins is well known to one of skill in the art and involves, by way of non-limiting examples, liquid fractionation, centrifugation, exchange columns, all of which is routine experimentation.

The plant protein homologues may be purified from the plant extracts. As such, in some embodiments, the at least one plant protein homologue may be a pure form.

In other embodiments, the plant protein homologues are not purified into their pure form from the plant extract or plant isolate. Rather, the plant extract or isolate comprising the at least one plant protein homologue is used as the source of the at least one plant protein homologue. As such, in some embodiments, the at least one plant protein homologue may be in the form of plant extract fractions. The plant fractions may be processed or further divided into isolates or additional fractions. In some embodiments, the plant fractions or isolates may be concentrated.

In some embodiments, the at least one plant protein homologue of a serum protein is produced recombinantly. Methods of recombinant production of plant proteins are known to those of skill in the art. Once produced, in some embodiments, the recombinant plant protein homologue may be isolated and/or purified by methods known to those of skill in the art.

Still another aspect of the present disclosure provides a kit comprising any of the herein disclosed cell culture medium supplements and instructions for mixing the supplement with a serum-free medium. In some embodiments, the serum-free medium is devoid of any animal proteins. In some embodiments, the serum-free medium is devoid of any animal components. The kit may further comprise additional components for cell culture.

Further aspects of the present disclosure provide use of a plant protein homologue of an animal protein in place of the animal protein in a cell culture medium supplement. In some embodiments, the animal protein is a serum protein. In some embodiments, the supplement is devoid of any animal proteins. In some embodiments, the supplement is devoid of any animal components. The plant protein homologue may be one or more of those disclosed herein.

In an aspect of the disclosure, there is provided an assay to measure animal cell or tissue growth promoting activity of a serum homolog. In some embodiments, the source of the cell or tissue is any edible species desired for consumption, which include, but are not limited to, livestock, poultry, fish, shellfish, crustaceans, and mollusk.

In some embodiments, the source of the cell or tissue is a livestock, e.g., cattle, sheep, pig, goat, lamb, horse, donkey, rabbit, and mule. In some embodiments, the source of the cell or tissue is an animal traditionally considered “game”, e.g., caribou, bear, boar, deer, elk, and moose. In some embodiments, the source of the cell or tissue is a poultry, e.g., chicken, duck, goose, guinea fowl, quail, and turkey. In some embodiments, the source of the cell or tissue is a fish, e.g., bass, carp, catfish, Chilean sea bass, cod, flounder, halibut, mahi mahi, monkfish, pike, perch, orange roughy, salmon, shad, snapper, swordfish, tilapia, trout, and tuna. In some embodiments, the source of the cell or tissue is a crustacean, e.g., crab, crayfish, lobster, prawn, and shrimp. In some embodiments, the source of the cell or tissue is a mollusk, e.g., clams, mussels, octopus, oysters, scallops, and squid.

In some embodiments, an assay is designed to determine whether a serum homologue functions as a substitute for a component of a predefined medium, wherein a serum homolog is tested by adding to a growth medium in which one or more predefined components of the medium are reduced, removed, or not added. In some embodiments, an assay is designed to determine whether a serum homolog increases cell growth and/or density when used as a supplement to a predefined medium.

In some embodiments, an assay system comprises an animal cell or tissue and a medium suitable for growth and/or development of the animal cell or tissue. In some embodiments, the medium comprises components in amounts sufficient for growth of the animal cell or tissue. In some embodiments, the medium comprises most but not all components in amounts that are sufficient for growth of the animal cell or tissue. In some embodiments, the medium is serum-free. In some embodiments, the medium comprises certain serum components but is deficient in other serum components. In some embodiments, one or more serum components are reduced, subtracted or eliminated, for example, by immunological (e.g., antibody) means.

In some embodiments, the medium or liquid base mix may comprise one or more elements of a base medium and supplements as described herein, e.g., salts, amino acids, vitamins, buffers, nucleotides, antibiotics, trace elements, antioxidants and glucose or an equivalent energy source, such that the medium is capable of be used as a serum-free complete medium.

Exemplary inorganic salts include, but are not limited to, potassium phosphate, calcium chloride (anhydrous), cupric sulfate, ferric nitrate, ferric sulfate, magnesium chloride (anhydrous), magnesium sulfate (anhydrous), potassium chloride, sodium bicarbonate, sodium chloride, sodium phosphate dibasic anhydrous, sodium phosphate monobasic, tin chloride and zinc sulfate. Exemplary organic salts include, but are not limited to, sodium bicarbonate or HEPES.

Exemplary sugars include, but are not limited to, dextrose, glucose, lactose, galactose, fructose and multimers of these sugars.

Exemplary antioxidants include, but are not limited to tocopherols, tocotrienols, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), flavonoids, isoflavones, lycopene, beta-carotene, selenium, ubiquinone, luetin, S-adenosylmethionine, glutathione, taurine, N-acetylcysteine, citric acid, L-carnitine, BHT, monothioglycerol, ascorbic acid, propyl gallate, methionine, cysteine, homocysteine, gluthatione, cystamine and cysstathionine, and glycine-glycine-histidine (tripeptide).

Exemplary trace elements, include, but are not limited to, copper, iron, zinc, manganese, silicon, molybdnate, molybdenum, vanadium, nickel, tin, aluminum, silver, barium, bromine, cadmium, cobalt, chromium, calcium, divalent cations, fluorine, germanium, iodine, rubidium, zirconium, or selenium. Additional trace metals are disclosed in WO 2006/004728.

In some embodiments, the medium or liquid base mix comprises an iron source or iron transporter. Exemplary iron sources include, but are not limited to, ferric and ferrous salts such as ferrous sulfate, ferrous citrate, ferric citrate, ferric nitrate, ferric sulfate, ferric ammonium compounds, such as ferric ammonium citrate, ferric ammonium oxalate, ferric ammonium fumarate, ferric ammonium malate and ferric ammonium succinate. Exemplary iron transporters include, but are not limited to, transferrin and lactoferrin.

In some embodiments, the medium or liquid base mix may further comprise a copper source or copper transporter (e.g., GHK-Cu). Exemplary copper sources include, but are not limited to, copper chloride and copper sulfate.

In some embodiments, the iron source or copper source is added to a serum replacement medium at a final concentration in the range of about 0.05 to 250 ng/ml, 0.05 to 100 ng/ml, from about 0.05 to 50 ng/ml, from about 0.05 to 10 ng/ml, from about 0.1 to 5 ng/ml, from about 0.5 to 2.5 ng/ml, or from about 1 to 5 ng/ml. It is further contemplated that the iron source or copper source is in a final concentration in the serum replacement of about 0.05, 0.1, 0.25, 0.35, 0.45, 0.5, 0.6, 0.7, 0.8, 1, 1.5, 2, 2.5, 3, 4, 5, 6, 7, 8, 9, or 10 ng/ml.

In some embodiments, the serum replacement or media supplement is added to a basic media. Standard basic media are known in the field of cell culture and commercially available. Examples of basic media include, but are not limited to, Dulbecco's Modified Eagle's Medium (DMEM), DMEM F12 (1:1), Iscove's Modified Dulbecco's Medium, Ham's Nutrient Mixture F-10 or F-12, Roswell Park Memorial Institute Medium (RPMI), MCDB 131, Click's medium, McCoy's 5A Medium, Medium 199, William's Medium E, and insect media such as Grace's medium and TNM-FH.

The serum replacement and medium supplement described herein are also contemplated for use in commercially available serum-free culture media. Exemplary serum-free media, include but are not limited to, AIM-V (Life Technologies, Carlsbad, Calif.), PER-C6 (Life Technologies, Carlsbad, Calif.), Knock-Out™ (Life Technologies), StemPro® (Life Technologies), CellGro® (Corning Life Sciences-Mediumtech Inc., Manassas, Va.).

Any of these media are optionally supplemented with salts (such as sodium chloride, calcium, magnesium, and phosphate), amino acids, vitamins, buffers (such as HEPES), nucleotides (such as adenosine and thymidine), antibiotics (such as gentamicin drug), trace elements (defined as inorganic compounds usually present at final concentrations in the micromolar range), antioxidants and glucose or an equivalent energy source. Any other necessary supplements may also be included at appropriate concentrations that would be known to those skilled in the art. The culture conditions, such as temperature, pH, and the like, will be apparent to the ordinarily skilled artisan.

It is contemplated that the medium compositions are packaged in unit forms. In one embodiment, the medium (serum replacement, medium supplement, complete medium or cryopreservation medium) is packaged in a volume of 10 ml, 50 ml, 100 ml, 500 ml or 1 L.

In an aspect of the disclosure, there is provided a method of culturing cells comprising the cell media supplement and/or medium disclosed herein.

It is contemplated that the media, e.g., serum replacement, media supplement, complete media, described herein is useful for culture of cells in vitro, preferably for cells that typically require serum supplements or defined media for adequate growth in vitro. Such cells include eukaryotic cells, such as mammalian cells, and insect cells. Mammalian cells contemplated to benefit from use of the serum replacement, complete media or media supplement include, without limitation, hamster, monkey, chimpanzee, dog, cat, cow/bull, pig, mouse, rat, rabbit, sheep and human cells. Insect cells include cells derived from Spodoptera frugiperda (caterpillar), Aedes aegypti (mosquito), Aedes albopictus (mosquito), Drosophila melanogaster (fruitfly), and Bombyx mori.

It is contemplated that the cells cultured with the serum replacement, complete media or media supplement, are immortalized cells (a cell line) or non-immortalized (primary or secondary) cells, and can be any of a wide variety of cell types that are found in vivo. Exemplary cell types include, but are not limited to, fibroblasts, keratinocytes, epithelial cells, ovary cells, endothelial cells, glial cells, neural cells, formed elements of the blood (e.g., lymphocytes, bone marrow cells), chondrocytes and other bone-derived cells, hepatocytes, pancreas cells, and precursors of these somatic cell types.

In some embodiments, the cells contemplated for use with the media are isolated from a mammalian subject. Cells isolated from a mammalian subject include, but are not limited to, pluripotent stem cells, embryonic stem cells, bone marrow stromal cells, hematopoietic progenitor cells, lymphoid stem cells, myeloid stem cells, lymphocytes, T cells, B cells, macrophages, endothelial cells, glial cells, neural cells, chondrocytes and other bone-derived cells, hepatocytes, pancreas cells, precursors of somatic cell types, and any carcinoma or tumor derived cell.

In some embodiments, the cells are a cell line. Exemplary cell lines include, but are not limited to, Chinese hamster ovary cells, including CHOK1, DXB-11, DG-44, and CHO/DHFR; monkey kidney CV1, COS-7; human embryonic kidney (HEK) 293; baby hamster kidney cells (BHK); mouse sertoli cells (TM4); African green monkey kidney cells (VERO); human cervical carcinoma cells (HELA); canine kidney cells (MDCK); buffalo rat liver cells (BRL 3A); human lung cells (W138); human hepatoma cells (Hep G2; SK-Hep); mouse mammary tumor (MMT); TRI cells; MRC 5 cells; FS4 cells; a T cell line (Jurkat), a B cell line, mouse 3T3, RIN, A549, PC12, K562, PER.C6®, SP2/0, NS-0, U205, HT1080, L929, hybridomas, tumor cells, and immortalized primary cells.

Exemplary insect cell lines, include, but are not limited to, Sf9, Sf21, HIGH FIVE™, EXPRESSF+®, S2, Tn5, TN-368, BmN, Schneider 2, D2, C6/36 and KC cells.

Additional cell types and cell lines are disclosed in WO 2006/004728, herein incorporated by reference. These cells include, but are not limited to, CD34+ hematopoietic cells and cells of myeloid lineage, 293 embryonic kidney cells, A-549, Jurkat, Namalwa, Hela, 293BHK cells, HeLa cervical epithelial cells, PER-C6 retinal cells (PER.C6), MDBK (NBL-I) cells, 911 cells, CRFK cells, MDCK cells, BeWo cells, Chang cells, Detroit 562 cells, HeLa 229 cells, HeLa S3 cells, Hep-G2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-I cells, Y-I cells, LLC-PK1 cells, PK (15) cells, GH1 cells, GH3 cells, L2 cells, LLC-RC 256 cells, MH1C1 cells, XC cells, MDOK cells, VSW cells, TH-I, B1 cells, or derivatives thereof, fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, fibroblast and fibroblast-like cell lines), TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, MR-90 cells, MRC-5 cells, WI-38 cells, WI-26 cells, MiC11 cells, CV-I cells, COS-I cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-I1 cells, NOR-IO cells, C3H/IOTI/2 cells, HSDM1C3 cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, NSO, NS1, Swiss/3T3 cells, Indian muntjac cells, SIRC cells, Cn cells, Jensen cells, COS cells and Sp2/0 cells, Mimic cells and/or derivatives thereof.

Cell culture conditions contemplated herein may be adapted to any culture substrate suitable for growing cells. Substrates having a suitable surface include tissue culture wells, culture flasks, roller bottles, gas-permeable containers, flat or parallel plate bioreactors or cell factories. Also contemplated are culture conditions in which the cells are attached to microcarriers or particles kept in suspension in stirred tank vessels.

Cell culture methods are described generally in the Culture of Animal Cells: A Manual of Basic Technique, 6.sup.th Edition, 2010 (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other reference texts include Creating a High Performance Culture (Aroselli, Hu. Res. Dev. Pr. 1996) and Limits to Growth (D. H. Meadows et al., Universe Publ. 1974). Tissue culture supplies and reagents are well-known to one of skill and are commercially available.

It is understood that the cells are placed in culture at densities appropriate for the particular cell line or isolated cell type used with the serum replacement, complete media or media supplement. In some embodiments the cells are cultured at 1×10³, 5×10³, 1×10⁴, 5×10⁴, 1×10⁵, 5×10⁵, 1×10⁶ or 5×10⁶ cells/ml.

In some embodiments, the cultured cells are fibroblasts. In an embodiment, the cells are bovine fibroblasts. In an embodiment, the cells are chicken fibroblasts.

Chicken embryonic fibroblasts are widely used for the production of viruses and vaccines. Together with chicken embryonic liver cells they are produced from specific pathogen-free (SPF) embryos and sold by Charles River Laboratories (Wilmington, Mass.) and other companies. While chicken liver cells show limited proliferation in culture, like their mammalian counterparts, chicken fibroblasts can undergo over 30 population doublings, producing about 2.6 ton of cells before spontaneously immortalizing without becoming tumorigenic. Spontaneously transformed chicken fibroblasts, such as UMNSAH/DF-1 (CRL-12203), can be bought directly from ATTC (Manassas, Va.). While the growth potential of fibroblast is excellent, the cells primarily form inedible connective tissue.

Chicken embryonic endothelium can be easily isolated but their growth potential is unknown and can be organ specific. Mouse micro-vascular cells can undergo 30 population doublings, while human cells seldom pass 12 population doublings. Chicken embryonic muscle cells (myocytes) can be similar isolated but have a very limited growth potential. Mouse and human cells seldom pass 12 population doublings. Myogenesis, the formation of new muscle tissue, is uncommon past the neonatal stage of life in most species. Small molecules can conceptually be used to modulate this behavior.

Numerous groups produced chicken embryonic stem cells (cESC) over the last decade. Cells are isolated from fertilized chicken eggs and are essentially immortal. Chicken induced pluripotent stem cells (ciPSC) were produced from quail embryonic fibroblasts by reprogramming factors OCT4, NANOG, SOX2, LIN28, KLF4, and C-MYC and more recently chicken fibroblasts using OCT4, KLF4, and C-MYC. Cells are essentially immortal but are genetically engineered.

Recently, mouse pluripotent stem cells were induced from fibroblasts using small molecules permitting the differentiation of multiple cell types, including myocytes, hepatocytes, and endothelial cells as well as complex embryoid bodies. Chemical induction of ciPSC offers an alternative approach to convert fibroblasts to other cell types.

In a more recent study, a combination of nine compounds that induced human fibroblasts to turn into cardiomyocytes were identified, while others used a seven compound combination to transform mouse cells. Considering many of the signaling pathways are conserved, a relatively similar combination could be used to transform chicken fibroblasts into myocytes.

As mentioned above, cell culture medium often contains fetal bovine serum (FBS) that provides attachment factors, fatty acids, growth factors, hormones, and albumin. FBS can usually be replaced with serum replacement (e.g. KO-serum) that is composed of amino acids, vitamins, and trace elements in addition to transferrin, insulin, and lipid-rich bovine serum albumin. While both transferrin and insulin are produced in bacteria using recombinant technology, albumin is usually animal derived. However, plant and bacteria-derived recombinant human albumin (e.g. Cellastim™) are available through several companies, including Sigma-Aldrich (St. Louis, Mo.).

Chicken fibroblast medium is traditionally composed of MI 99 medium supplemented with 10% FBS, tryptose phosphate and glutamine. However, serum-free medium for the growth of mammalian fibroblasts is now readily available. Medium is composed of M199 supplemented with 0.5 mg/mL albumin, 0.6 μM linoleic acid, 0.6 μg/mL lecithin, 5 ng/niL bFGF, 5 ng/niL EGF, 30 pg/mL TGFpi, 7.5 mM glutamine, 1 μg/mL hydrocortisone, 50 μg/mL ascorbic acid, and 5 μg/mL insulin. This medium PCS-201-040 is available from ATCC (Manassas, Va.) and is reported to support 4-fold faster proliferation of human fibroblasts. Chicken hepatocytes are similarly supported by a serum-free culture medium designed for human and mouse hepatocytes. Medium is composed of Williams E basal medium supplemented with albumin, insulin, transferrin, and hydrocortisone.

Chicken and bovine anchorage-independent fibroblasts are differentiated into anchorage-independent adipocytes by standard differentiation protocols. FMT-SCF-2 (chicken non-adherent fibroblasts) and FMT-SBF-1 (bovine non-adherent fibroblast) were grown in adipogenesis medium containing 200 μM oleic acid together with a PPARgamma agonists. A synthetic inhibitor (Rosiglitazone) and a natural inhibitor (Pristanic acid) were both tested.

Perfused culture medium can also include an oxygen carrier. Hemoglobin based oxygen carriers include hemoglobin derivatives either recombinant or chemically modified, encapsulated hemoglobin or modified (e.g. cross-linked) red blood cells. Alternatives include Perfluorocarbon based alternatives such as those developed in Nahmias et al. (The FASEB Journal, 20(14): 2531-2533).

It should be noted that normally, primary fibroblast cells are capable of a limited cell division, and thus undergo cellular senescence after about 30 population doublings (e.g., 10 passages). Methods of generating immortalized fibroblastoid cell lines include genetic manipulation by introduction of a telomerase gene, or SV40, or HPVE6/E7 gene using known methods.

It is contemplated that other avian fibroblast cells are also suitable, e.g., duck, goose, and quail fibroblast cells.

Another aspect of the present disclosure provides a method of producing cultured meat by culturing cells in any of the herein disclosed cell culture medium and producing meat from the cultured cells.

In some embodiments, the cells are from edible animals. In some embodiments, the animal is a livestock animal, e.g., cattle, sheep, pig, goat, lamb, horse, donkey, rabbit, and mule. In some embodiments, the animal is an animal traditionally considered “game”, e.g., caribou, bear, boar, deer, elk, and moose. In some embodiments, the animal is a poultry, e.g., chicken, duck, goose, guinea fowl, quail, and turkey. In some embodiments, the animal is a fish, e.g., bass, carp, catfish, Chilean sea bass, cod, flounder, halibut, mahi mahi, monkfish, pike, perch, orange roughy, salmon, shad, snapper, swordfish, tilapia, trout, and tuna. In some embodiments, the animal is a crustacean, e.g., crab, crayfish, lobster, prawn, and shrimp. In some embodiments, the animal is a mollusk, e.g., clams, mussels, octopus, oysters, scallops, and squid.

In some embodiments, the cells are fibroblasts. In an embodiment, the fibroblasts include, but are not limited to, bovine fibroblasts and chicken fibroblasts. In an embodiment, the fibroblasts are bovine fibroblasts. In an embodiment, the fibroblasts are chicken fibroblasts.

Yet another aspect of the present disclosure provides cultured meat produced by the above method.

Still yet another aspect of the present disclosure provides a cell culture medium devoid of any animal proteins and/or animal components and methods for producing said cell culture medium.

The cell culture medium may comprise a serum-free medium and any of the herein disclosed cell culture medium supplements comprising at least one plant protein homologue. The cell culture medium may be devoid of any animal components and/or devoid of any animal proteins. The at least one plant protein homologue may be a homologue of an animal protein. The at least one plant protein homologue may be a homologue of a serum protein. Examples of such plant protein homologues of serum proteins are disclosed above and herein.

In some embodiments, the serum-free medium is a base physiological buffer, and is devoid of animal contaminants, human contaminants, or any antibiotic(s). In some embodiments, the serum-free medium is a base physiological buffer and is devoid of any animal proteins. In some embodiments, the serum-free medium is a base physiological buffer and is devoid of any animal components.

Exemplary serum-free medium, include but are not limited to, AIM-V (Life Technologies, Carlsbad, Calif.), PER-C6 (Life Technologies, Carlsbad, Calif.), Knock-Out™ (Life Technologies), StemPro® (Life Technologies), CellGro® (Corning Life Sciences-Mediumtech Inc., Manassas, Va.).

Any of these media are optionally supplemented with salts (such as sodium chloride, calcium, magnesium, and phosphate), amino acids, vitamins, buffers (such as HEPES), nucleotides (such as adenosine and thymidine), antibiotics (such as gentamicin drug), trace elements (defined as inorganic compounds usually present at final concentrations in the micromolar range), antioxidants and glucose or an equivalent energy source. Any other necessary supplements may also be included at appropriate concentrations that would be known to those skilled in the art. The culture conditions, such as temperature, pH, and the like, will be apparent to the ordinarily skilled artisan.

In some embodiments, the cell culture medium may comprise one or more elements of a base medium and supplements as described herein, e.g., salts, amino acids, vitamins, buffers, nucleotides, antibiotics, trace elements, antioxidants and glucose or an equivalent energy source, such that the cell culture medium is capable of be used as a serum-free complete medium.

Exemplary inorganic salts include, but are not limited to, potassium phosphate, calcium chloride (anhydrous), cupric sulfate, ferric nitrate, ferric sulfate, magnesium chloride (anhydrous), magnesium sulfate (anhydrous), potassium chloride, sodium bicarbonate, sodium chloride, sodium phosphate dibasic anhydrous, sodium phosphate monobasic, tin chloride and zinc sulfate. Exemplary organic salts include, but are not limited to, sodium bicarbonate or HEPES.

Exemplary sugars include, but are not limited to, dextrose, glucose, lactose, galactose, fructose and multimers of these sugars.

Exemplary antioxidants include, but are not limited to tocopherols, tocotrienols, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman carboxylic acid), butylated hydroxyanisole (BHA), butylated hydroxytoluene (BHT), flavonoids, isoflavones, lycopene, beta-carotene, selenium, ubiquinone, luetin, S-adenosylmethionine, glutathione, taurine, N-acetylcysteine, citric acid, L-carnitine, BHT, monothioglycerol, ascorbic acid, propyl gallate, methionine, cysteine, homocysteine, gluthatione, cystamine and cysstathionine, and glycine-glycine-histidine (tripeptide).

Exemplary trace elements, include, but are not limited to, copper, iron, zinc, manganese, silicon, molybdnate, molybdenum, vanadium, nickel, tin, aluminum, silver, barium, bromine, cadmium, cobalt, chromium, calcium, divalent cations, fluorine, germanium, iodine, rubidium, zirconium, or selenium. Additional trace metals are disclosed in WO 2006/004728.

In some embodiments, the cell culture medium comprises an iron source or iron transporter. Exemplary iron sources include, but are not limited to, ferric and ferrous salts such as ferrous sulfate, ferrous citrate, ferric citrate, ferric nitrate, ferric sulfate, ferric ammonium compounds, such as ferric ammonium citrate, ferric ammonium oxalate, ferric ammonium fumarate, ferric ammonium malate and ferric ammonium succinate. Exemplary iron transporters include, but are not limited to, transferrin and lactoferrin.

In some embodiments, the cell culture medium may further comprise a copper source or copper transporter (e.g., GHK-Cu). Exemplary copper sources include, but are not limited to, copper chloride and copper sulfate.

In some embodiments, the iron source or copper source is added to the cell culture medium at a final concentration in the range of about 0.05 to 250 ng/ml, 0.05 to 100 ng/ml, from about 0.05 to 50 ng/ml, from about 0.05 to 10 ng/ml, from about 0.1 to 5 ng/ml, from about 0.5 to 2.5 ng/ml, or from about 1 to 5 ng/ml.

It is contemplated that the cell culture medium is packaged in unit forms. In one embodiment, the cell culture medium is packaged in a volume of 10 ml, 50 ml, 100 ml, 500 ml or 1 L.

The cell culture medium may further comprise other components, assuming said components are devoid of any animal components and/or animal proteins.

Also disclosed are methods of producing the cell culture media described herein. The method for producing a cell culture medium may comprise admixing a serum-free base medium and a cell culture medium supplement, wherein the cell culture medium is devoid of any animal proteins and/or animal components. The cell culture medium supplement comprises at least one plant protein homologue of an animal protein. In some embodiments, the animal protein is a serum protein. The plant protein homologue may be one or more of those disclosed herein.

Exemplary serum-free media are provided herein.

In some embodiments, the method further comprising adding one or more additional components, assuming said components are devoid of any animal components. Exemplary additional components are provided herein.

The disclosure further provides for a kit comprising a cell culture medium as described herein and instructions for use. In some embodiments, the cell culture medium is packaged in a container with a label affixed to the container or included in the package that describes use of the compositions for use in vitro, in vivo, or ex vivo. Exemplary containers include, but are not limited to, a vessel, vial, tube, ampoule, bottle, flask, and the like. It is further contemplated that the container is adapted for packaging the media, e.g., serum replacement, media supplement or cryopreservation media in liquid or frozen form. It is contemplated that the container is made from material well-known in the art, including, but not limited to, glass, polypropylene, polystyrene, and other plastics. In various aspects, the compositions are packaged in a unit dosage form. The kit optionally includes a device suitable for combining the serum replacement, media supplement or cryopreservation media with a basic media, and alternatively combining the media with additional growth factors. In various aspects, the kit contains a label and/or instructions that describes use of the media for cell culture or cryopreservation.

All applications and all documents cited herein or during their prosecution (“appin cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The following examples are offered by way of illustration and not by way of limitation.

### Example 1: Isolation and Purification of Plant Albumin

Isolation of plant albumins is well known to one of skill in the art and involves liquid fractionation, centrifugation, exchange columns, all of which is routine experimentation. In this study, two species of potato (white and red) were purchased. Each species was either juiced (juicer) or blended (blender). Potato albumin was extracted, liquid fraction extraction of raw white and red potatoes was executed by either fruit juicer or kitchen blender. In the latter, potato was chopped and added to the blender with excess of water. Blended material was sieved/filtered using two layers of gauze, to obtain the liquid fraction. Samples were spun down at 11,000 rpm for 10 min at 4° C. to remove insoluble. The supernatant (albumin fraction) was collected, while pellets were discarded.

The supernatant sample was adjusted to pH 3 according to Jirgensons (1946, Journal of Polymer Science, 1(6): 484-494), and then spun down again to remove insoluble protein impurities. Supernatants were dialyzed over night against 20 mM TRIS-HCl, pH 8. To get rid of extra salt in the samples, each was dialyzed against buffer A (see below)—1 lit for 1 h and then 1 lit ON (cold room, with strearing).

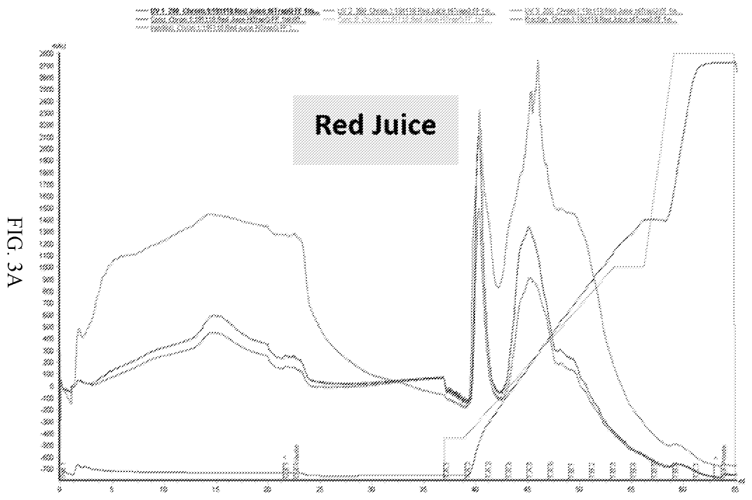

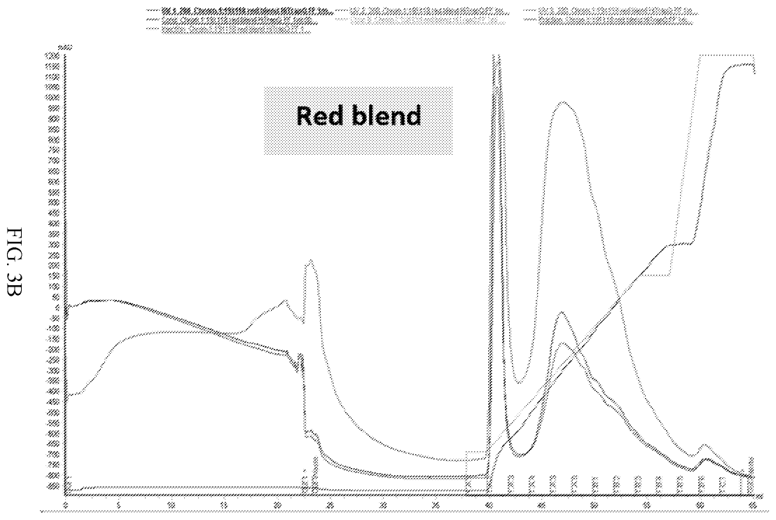

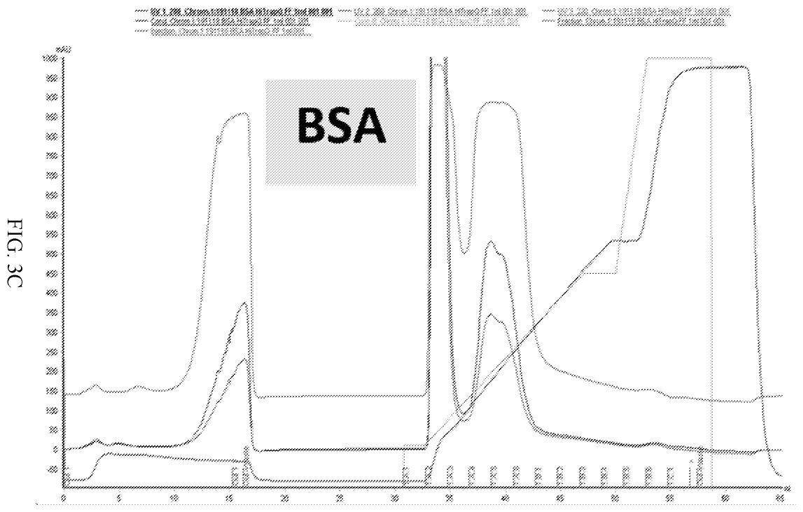

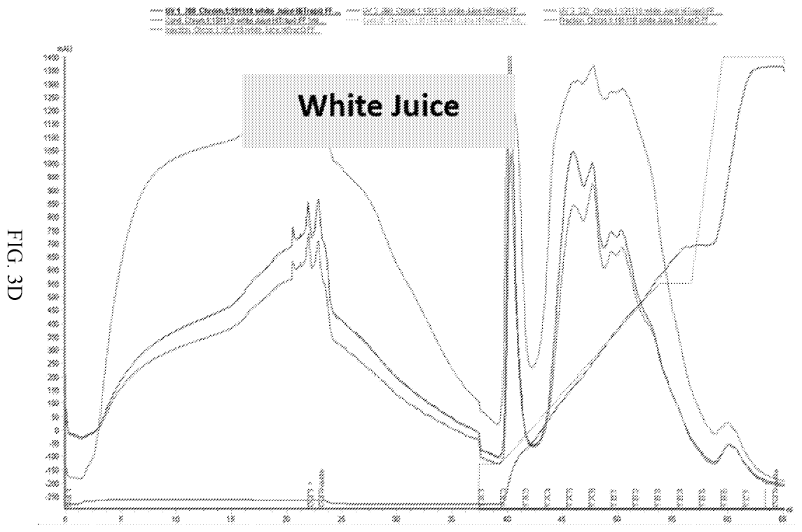

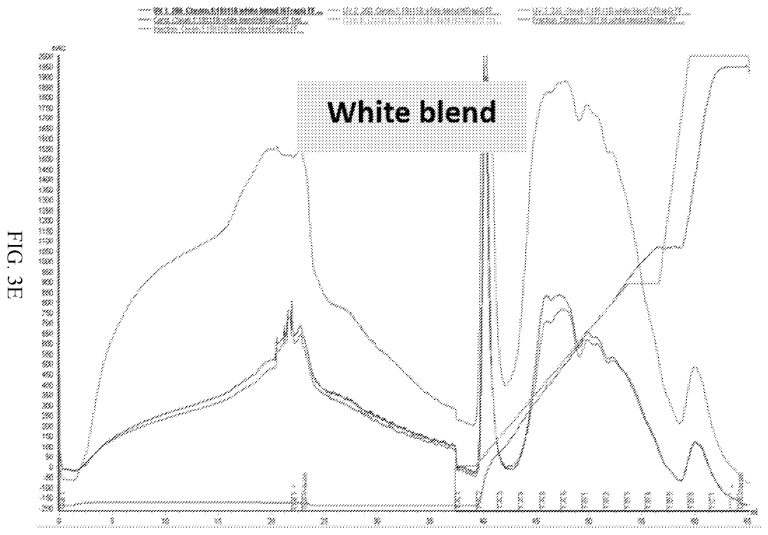

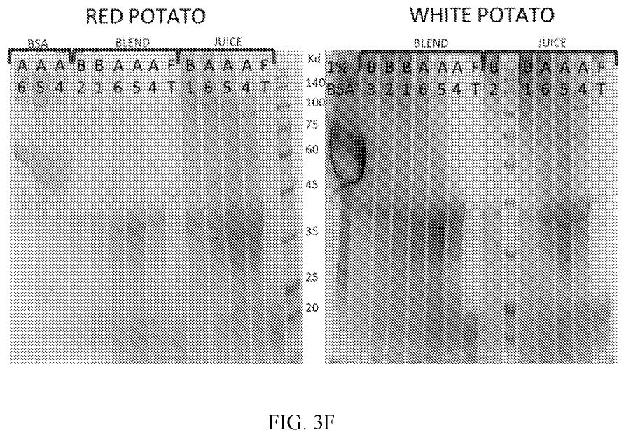

The four samples were loaded on an anion exchange column according to pI ˜5 to separate albumin fraction. Sample loading buffer was 20 mM TRIS HCl, pH 8 and releasing buffer was 20 mM TRIS HCl, 1 M NaCl, pH 8. 8 μl samples from the extracted fractions were loaded on a 4-20% SDS-PAGE for protein purity indication and analysis (FIG. 3F), 1% BSA was loaded as a control. Relevant fractions were combined and a few μg of each were MS analyzed to confirm BSA-like protein content (FIGS. 3A-3E).

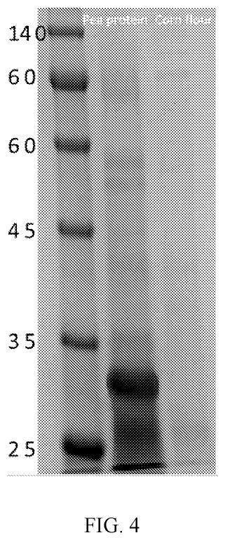

Five (5) grams of pea protein isolate were suspended in 50 ml 10 mM CaCl₂, 10 mM MgCl₂, pH 8 according to Nadal et al., J. Agric. Food Chem. 2011, 59, 2752-2758. Samples were vortexed at room temperature for 30 minutes, then spun down at 11,000 rpm for 15 minutes at 4° C. and supernatant was collected and ran on SDS-PAGE (FIG. 4).

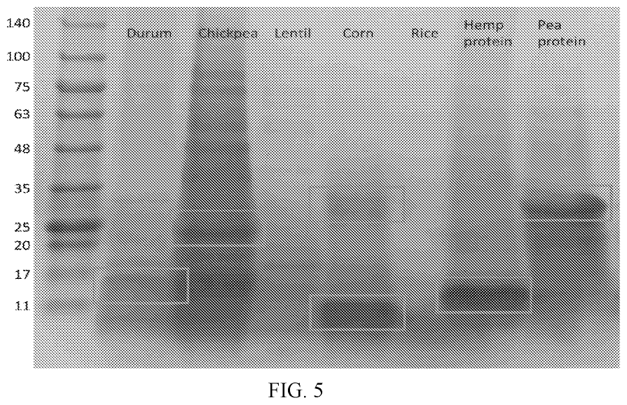

Powders of five plant flours (durum, chickpea, lentil, corn, rice) and two commercial plant protein isolates (hemp, pea) were liquid fractionated to separate lipids from DNA and RNA and from proteins. Water fraction of protein extraction was collected and ran on an SDS-PAGE. Chickpea, Corn, Hemp and Pea samples contain protein bands which correspond in size to formerly reported albumin proteins (FIG. 5). The boxed bands were isolated from gel and sent to MS analysis for further identification.

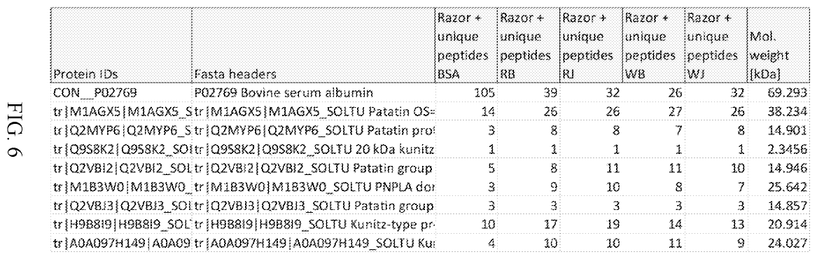

The results of MS analysis of four potato extractions from two potato types (Red or White) are shown in FIG. 6. Each was liquidized either in a fruit juicer (J) or blended with water (B) and sieved to remove fiber fraction. BSA was analyzed as a positive control. All potato samples were shown to contain significant amounts of BSA-like proteins. Identified proteins of Solanum tubrosum include albumins (patatins) (UniProtKB: M1AGX5, Q2MYP6, Q2VBI2, Q2VBJ3, A0A097H149), patatin-like phospholipase domain-containing proteins (PNPLAs) (UniProtKB: M1B3W0), and proteinase inhibitors (Kunitz-type proteinase inhibitor group A1 (UniProtKB: H9B819); 20 kDa Kunitz-type proteinase inhibitor (UniProtKB: Q9S8K2).

### Example 2: Attachment of Cultured Cells in Serum-Free Medium

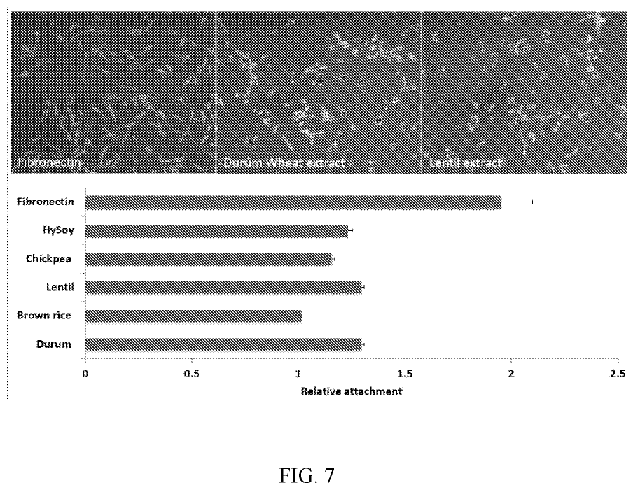

It was hypothesized that soluble plant ECM like proteins would be able to support attachment of cultured cells in the absence of serum and animal-derived ECM proteins. Protein extraction from chickpea, lentil, durum and brown rice flours was done by suspending them in PBS, shaking at room temperature for 24 Hours, spinning down at 13,000×g and filtering using a 0.22 μm syringe filter. Primary chicken fibroblasts cultured in DMEM/F12 supplemented with 15% FBS were trypsinized, washed and reseeded in serum free medium supplemented with 1:50 or 1:100 dilutions of the above protein constructs. The extent of attachment was assessed after 8 hours, using Sulforhodamin B staining (Vichai and kirtikara, Nat Protoc. 2006; 1(3): 1112-6). Results demonstrate cell attachment in soy, chickpea, lentil, rice and wheat extracts (FIG. 7).

### Example 3: Preparation of Complete Protein Bulks

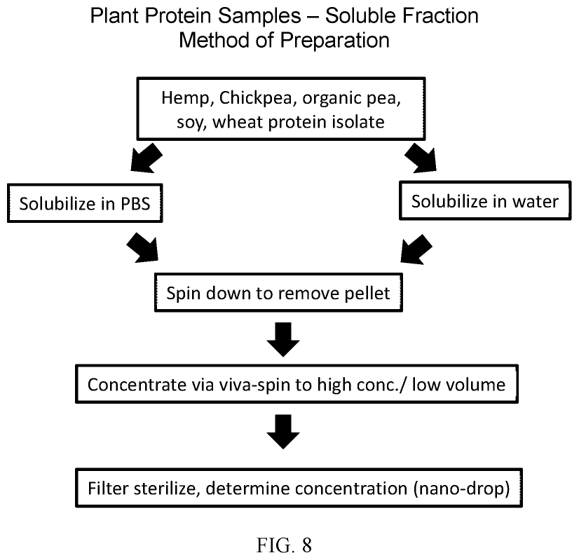

To prepare complete protein bulks as a replacement of bovine serum albumin (BSA), the protein powder from different plant source was first mixed with either water or saline (PBS) on a stirrer. As measured for different plant protein sources, the recovery % of proteins ranged from 10%-15%. Next, the mixture was centrifuged using Sorval with high speed to remove the insoluble fraction, and the soluble fraction was then filtered and concentrated using either centricons, amplicons, or holo-fiber with a cutoff of 10 kilodalton (FIG. 8). Afterwards, the mix was stored at +4° C. to use in a period of 1-2 months.

### Example 4: Albusorb Purification of Soy Protein

To purify soy protein (water soluble fraction), 50 mg of AlbuSorb™ powder was placed in a spin-tube/microfuge tube. 400 μl of Binding Buffer BB1 was added to the tube to condition the AlbuSorb™ powder. After mixing the contents thoroughly either manually or by vortexing for 3 min, the tube was then centrifuged for 2 minutes at 3000 rpm. The supernatant was discarded. Another 400 μl of BB1 Buffer was added to the tube again, followed by mixing and centrifuging. The supernatant was again discarded.

As a requirement for albumin binding, 250 μl of BB1 Buffer was added, followed by addition of 25 μl of the serum. The tube was then placed on a rotating shaker for 10 minutes. Afterwards, the tube was centrifuged for 4 minutes at 10,000 rpm. The resultant supernatant contains serum proteins minus albumin. Optionally, the pellet (mostly albumin) can be eluted with 200 μl of stripping buffer (0.2M Tris+0.5M NaCl, pH 10 by mixing on a shaker for 10 min) and centrifuged for 4 minutes at 10,000 rpm.

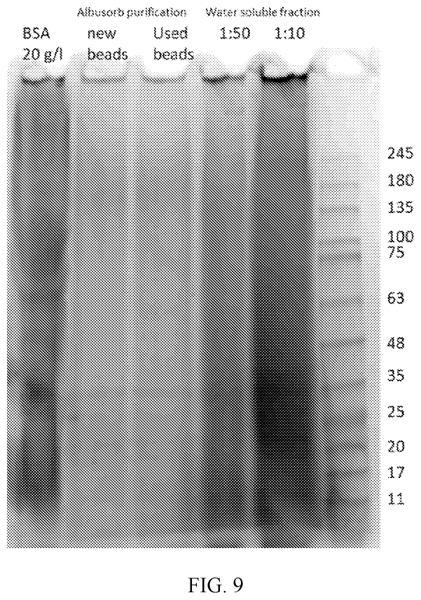

The supernatant was collected and ran on an SDS-PAGE. FIG. 9 shows soy protein (water soluble fraction) before and after Albusorb purification. It is noted that when observing proteins on SDS-PAGE (4-15%), other proteins migrated to the same region as albumin, and may not have be fully resolved.

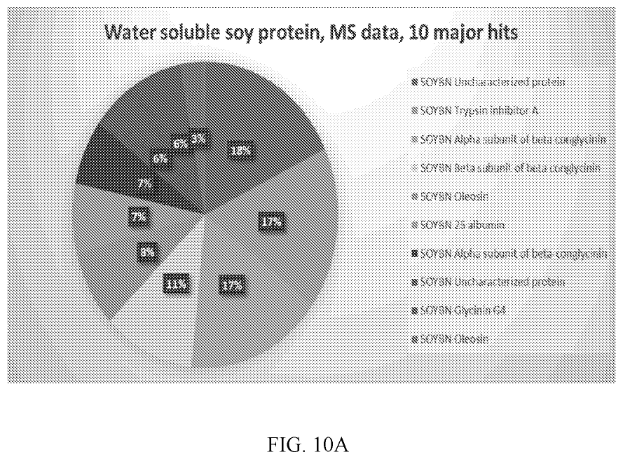

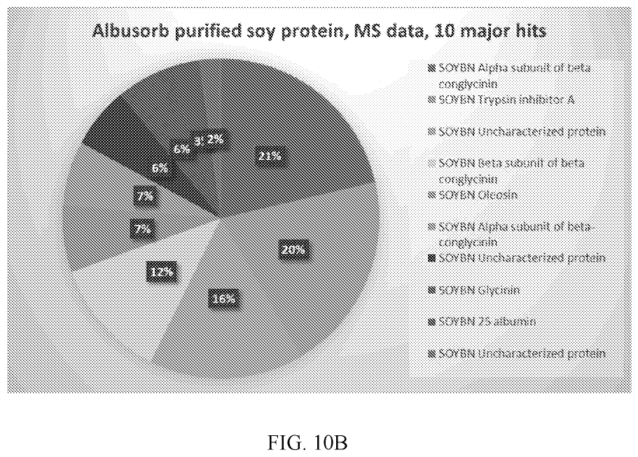

Water soluble soy protein and Albusorb purified soy protein were sent to MS analysis for further identification. FIG. 10A and FIG. 10B show the top 10 protein groups in soy water soluble fraction before and after Albusorb purification, respectively.

### Example 5: MS Analysis of Chickpea Proteins

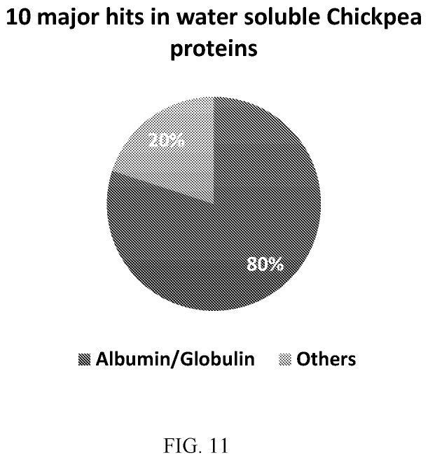

10 μg of chickpea protein dissolved in 8 M urea, 25 mM Tris-HCl, pH 8.0, 10 mM dithiothreitol (DTT) were alkylated with 55 mM iodoacetamide for 30 min at room temperature. The sample was diluted 8-fold with Tris-HCl, pH 8.0. 0.3 μg trypsin (sequencing grade, from Promega Corp., Madison, Wis., USA) was then added to the sample and digestion was performed overnight at 37° C. The tryptic peptides were desalted on C18 Stage tips (Rappsilber J, Mann M, Ishihama Y. Protocol for micro-purification, enrichment, pre- fractionation and storage of peptides for proteomics using StageTips. Nat Protoc. 2007; 2(8): 1896-906). A total of 0.8 μg of peptides (by O.D. 280 nm) were injected into the mass spectrometer (MS) for analysis (FIG. 11).

MS analysis was performed using a Q Exactive Plus mass spectrometer (Thermo Fisher Scientific) coupled on-line to a nanoflow UHPLC instrument (Ultimate 3000 Dionex, Thermo Fisher Scientific). Eluted peptides were separated over a 90-min gradient run at a flow rate of 0.2 μl/min on a reverse phase 25-cm-long C18 column (75 um ID, 2 um, 100 Å, Thermo PepMap® RSLC). The survey scans (380-2,000 m/z, target value 3E6 charges, maximum ion injection times 200 ms) were acquired and followed by higher energy collisional dissociation (HCD) based fragmentation (normalized collision energy 25). A resolution of 70,000 was used for survey scans and up to 15 dynamically chosen most abundant precursor ions were fragmented (isolation window 1.6 m/z). The MS/MS scans were acquired at a resolution of 35,000 (target value 2E5 charges, maximum ion injection times 121 ms). Dynamic exclusion was 15 sec.

MS data were processed using the MaxQuant computational platform, version 1.6.6.0 (Cox, J. & Mann, M. MaxQuant enables high peptide identification rates, individualized p.p.b-range mass accuracies and proteome-wide protein quantification, Nat. Biotechnol. 26: 1367-1372 (2008)). Peak lists were searched against the Cicer arietinum database from Uniprot, containing 57,497 entries. The search included cysteine carbamidomethylation as a fixed modification and oxidation of methionine and N-terminal acetylation as variable modifications. Peptides with minimum of seven amino-acid length were considered and the required FDR was set to 1% at the peptide and protein level. Protein identification required at least 2 unique or razor peptides.

### Example 6: Plant Proteins to Replace Bovine Serum Albumin

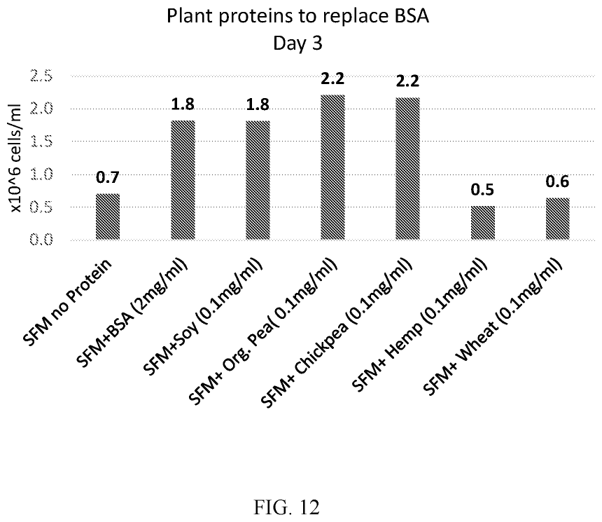

The effect of different plant water soluble fraction proteins was tested on chicken fibroblast cells using a special serum free supplement that was depleted from BSA. Chicken fibroblasts adapted to the suspension culture were seeded in 0.3 million/ml in a total volume of 20 ml in flasks. Cell culture flasks were kept in a shaker incubator with 100 rpm, 39° C., and 5% CO2. On day 3, 1 ml samples from each flask were counted using automatic cell counter (Cellaca) using AOPI to determine the living cells from dead cells. Living cell-counts are presented in FIG. 12. It was shown that 0.1 mg/ml was enough to replace the animal protein (BSA). However, Hemp and Wheat proteins didn't support the growth of the chicken fibroblasts at this concentration.

### Example 7: Plant Proteins and Dose Dependent Effect

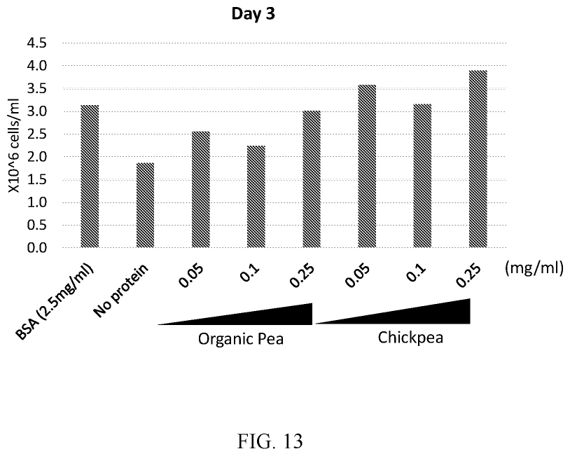

Gradient concentration of both chickpea and organic pea proteins was tested on chicken fibroblasts in a suspension culture to replace animal protein (BSA) in a serum free medium. Chicken fibroblasts adapted to the suspension culture were seeded in 0.3 million/ml in a total volume of 20 ml in flasks. Cell culture flasks were kept in a shaker incubator with 100 rpm, 39° C., and 5% CO2. As shown in FIG. 13, different ranges of protein concentrations still work almost as well as or better than BSA. Cell counts were done on day 3 using automatic cell counter (Cellaca) using APOI staining to eliminate the dead cells from our counts.

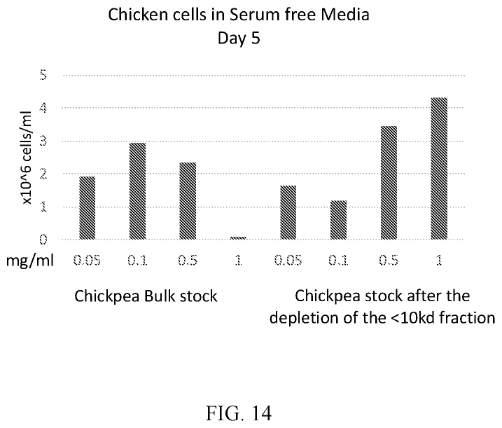

Increasing the concentration of chickpea or soy proteins (data not shown) resulted with a toxicity that is dose dependent (FIG. 14). Depletion of the <10 kilodalton fraction from the protein mix using centricons significantly eliminated this toxicity. Chicken fibroblasts adapted to the suspension culture were seeded in 0.3 million/ml in a total volume of 20 ml in flasks. Cell culture flasks were kept in a shaker incubator with 100 rpm, 39° C., and 5% CO2. On day 5, 1 ml samples from each flask were counted using automatic cell counter (Cellaca) using AOPI to determine the living cells from dead cells. Living cell-counts are presented in FIG. 14.

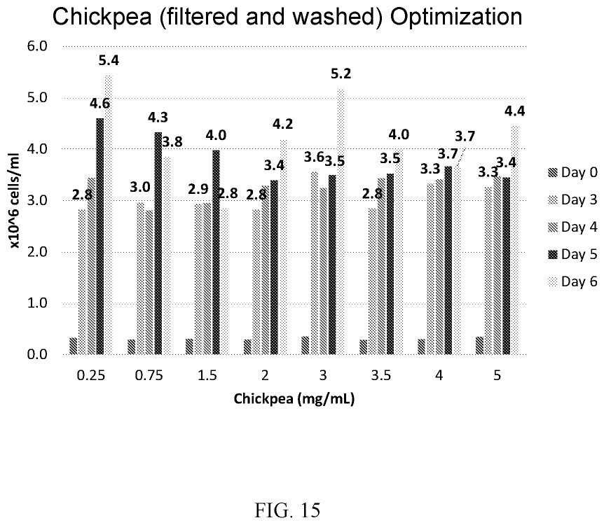

Gradient concentration of chickpea that were washed 3 times on a hollow fiber of 10 kilodalton cutoff. 5 mg/ml of chickpea had no toxicity on chicken cells (FIG. 15). Chicken fibroblasts adapted to the suspension culture were seeded in 0.3 million/ml in a total volume of 20 ml in flasks. Cell culture flasks were kept in a shaker incubator with 100 rpm, 39° C., and 5% CO2. On day 3-day 6, 1 ml samples from each flask were counted using automatic cell counter (Cellaca) using AOPI to determine the living cells from dead cells. Living cell-counts are presented FIG. 15.

Although the present invention and its advantages have been described in detail, it should be understood that various changes, substitutions and alterations can be made herein without departing from the spirit and scope of the invention as defined in the appended claims.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A cell culture medium supplement comprising at least one plant protein homologue of a serum protein, wherein said supplement is devoid of any serum proteins.

2. (canceled)

3. The cell culture medium supplement of claim 1, wherein said supplement is essentially devoid of any animal serum-derived component.

4. The cell culture medium supplement of claim 1, wherein the at least one plant protein homologue comprises the water soluble fraction of a plant protein isolate, wherein the water soluble fraction comprises plant albumins and globulins.

5. (canceled)

6. The cell culture medium supplement of claim 1, wherein said at least one plant protein homologue is a homologue of a serum albumin, a serum catalase, a serum superoxide dismutase, a serum transferrin, a serum fibronectin, a serum vitronectin, a serum insulin, a serum hemoglobin, a serum aldolase, a serum lipase, a serum transaminase, a serum aminotransferase, a serum fetuin, or a combination thereof.

7. The cell culture medium supplement of claim 6, wherein said at least one plant protein homologue is a plant albumin, a plant catalase, a plant superoxide dismutase, a plant transferrin, a plant fibronectin, a plant vitronectin, a plant insulin, a plant leghemoglobin, a plant aldolase, a plant lipase, a plant transaminase, a plant aminotransferase, a plant cystatin, or a combination thereof.

8. The cell culture medium supplement of claim 7, wherein the at least one plant protein homologue comprises a plant albumin, a plant catalase, a plant fibronectin, and a plant insulin.

9. The cell culture medium supplement of claim 7, wherein said at least one plant protein homologue is a plant albumin.

10. The cell culture medium supplement of claim 9, wherein said plant albumin is a chickpea albumin, a hempseed albumin, a lentil albumin, a pea albumin, a soy albumin, a wheat albumin or a potato albumin.

11. (canceled)

12. The cell culture medium supplement of claim 9, wherein said plant albumin is from the water soluble fraction of a plant protein isolate.

13-16. (canceled)

17. The cell culture medium supplement of claim 9, wherein said plant albumin is present in the cell culture medium supplement at a concentration such that when the cell culture medium supplement is added to a cell culture medium the plant albumin has a final concentration of about 0.01% to about 10% by weight in the cell culture medium.

18. The cell culture medium supplement of claim 7, wherein said at least one plant protein homologue is a plant catalase.

19. The cell culture medium supplement of claim 18, wherein said plant catalase is an Arabidopsis catalase, a cabbage catalase, a cucumber catalase, a cotton catalase, a potato catalase, a pumpkin catalase, a spinach catalase, a sunflower catalase, a tobacco catalase or a tomato catalase.

20-21. (canceled)

22. The cell culture medium supplement of claim 18, wherein said plant catalase is present in the cell culture medium supplement at a concentration such that when the cell culture medium supplement is added to a cell culture medium the plant catalase has a final concentration of about 1 ng/ml to about 100 ng/ml in the cell culture medium.

23. The cell culture medium supplement of claim 7, wherein said at least one plant protein homologue is a plant fibronectin.

24. The cell culture medium supplement of claim 23, wherein said plant fibronectin is a bean fibronectin, a chickpea fibronectin, a lentil fibronectin, a rice fibronectin, a soy fibronectin, a tobacco fibronectin or a wheat fibronectin.

25-26. (canceled)

27. The cell culture medium supplement of claim 23, wherein said plant fibronectin is present in the cell culture medium supplement at a concentration such that when the cell culture medium supplement is added to a cell culture medium the plant fibronectin has a final concentration of about 0.1 μg/ml to about 100 μg/ml in the cell culture medium.

28. The cell culture medium supplement of claim 7, wherein said at least one plant protein homologue is a plant insulin.

29. The cell culture medium supplement of claim 28, wherein said plant insulin is glucokinin, charantin, or corosolic acid.

30. The cell culture medium supplement of claim 28, wherein said plant insulin is present in the cell culture medium supplement at a concentration such that when the cell culture medium supplement is added to a cell culture medium the plant insulin has a final concentration of about 0.05 μg/ml to about 10 μg/ml in the cell culture medium.

31. (canceled)

32. A cell culture medium comprising a serum-free medium and the cell culture medium supplement according to claim 1, wherein said serum-free medium is essentially devoid of any animal serum-derived components.

33-34. (canceled)

35. A method of producing cultured meat, comprising culturing cells in the cell culture medium of claim 32, and producing meat from the cultured cells.

36. The method of claim 35, wherein said cells are from edible animals, wherein the edible animal is livestock, game, poultry, fish, or crustacean.

37. (canceled)

38. The method of claim 35, wherein said cells are fibroblasts.

39. The method of claim 38, wherein said fibroblasts are bovine fibroblasts or chicken fibroblasts.

40. Cultured meat produced by the method of claim 35.

41-45. (canceled)


SEQUENCE LISTING (43 sequences)

SEQ ID NO: 1 - protein, 130 aa, Pisum sativum
MASVKLASLIVLFATLGMFLTKNVGAASCNGVCSPFEMPPCGTSACRCIPVGLVIGYCRNPSGVFLRTNDEHPNLCESDADCRKKGSGKFCGHYPNPGIEYGWCFASKSEAEDFFSKITQKDLLKSVSTA

SEQ ID NO: 2 - protein, 130 aa, Pisum sativum
MASVKLASLIVLFATLGMFLTKNVGAASCNGVCSPFEMPPCGSSACRCIPVGLLIGYCRNPSGVFLKGNDEHPNLCESDADCKKKGSGNFCGHYPNPDIEYGWCFASKSEAEDVFSKITPKDLLKSVSTA

SEQ ID NO: 3 - protein, 130 aa, Pisum sativum
MASVKLASLMVLFATLGMFLTKNVGAASCNGVCSPFEMPPCGSSACRCIPVGLVVGYCRHPSGVFLRTNDEHPNLCESDADCRKKGSGNFCGHYPNPDIEYGWCFASKSEAEDFFSKITQKDLLKSVSTA

SEQ ID NO: 4 - protein, 130 aa, Pisum sativum
MASVKLASLIVLFATLGMFLTKNVGAISCNGVCSPFDIPPCGSPLCRCIPAGLVIGNCRNPYGVFLRTNDEHPNLCESDADCRKKGSGTFCGHYPNPDIEYGWCFASKSEAEDVFSKITPKDLLKSVSTA

SEQ ID NO: 5 - protein, 130 aa, Pisum sativum
MASVKLASLIVLFATLGMFLTKNVGAASCNGVCSPFEMPPCGTSACRCIPVGLVVGYCRNPSGVFLRTNDEHPNLCESDADCRKKGSGNFCGHYPNPDIEYGWCFASKSEAEDFFSKITPKDLLKSVSTA

SEQ ID NO: 6 - protein, 130 aa, Pisum sativum
MASVKLASLIVLFATLGMFLTKNVGAASCNGVCSPFEMPPCGTSACRCIPVGLFIGYCRNPSGVFLKANDEHPNLCESDADCRKKGSGNFCGHYPNPDIEYGWCFASKSEAEDFFSKITPKDLLKSVSTA

SEQ ID NO: 7 - protein, 134 aa, Medicago truncatula
MAYFKLASLAVFLLATFLMFPTKNVEAQSCSGAVCIRFNTECDAGCYCHTAGTEQTGVCRPNVDGMEMEERHPYLCQSHDECNKKGSGSFCARSPNSDNKNGWCFASFSEAQEYFKFTAKYKFKRDFLKMPITA

SEQ ID NO: 8 - protein, 134 aa, Medicago truncatula
MAYFKLASLAVFLLATFLMFPTKNVEAQSCSGAVCIRFNTECDAGCYCHTAGTEQTGVCRPNVDGMEMEERHPYLCQSHDECNKKGSGSFCARFPNFDNKNGWCFASFSEAQEYFKFTAKYKFKRDFLKMPITA

SEQ ID NO: 9 - protein, 140 aa, Medicago truncatula
MTYVKLAPLAVFLLAAFLIFPMKKVEADKCGAYCPYPRLYCSGDCDCEPFIASLPPRLNFKCVTPHSSAELKKKVEEQPKLCWSHTECTEKGSGNYCARFPNSNLKYGLCFPSISEAVNTFKMASSLKFEKDFLKMSLPA

SEQ ID NO: 10 - protein, 119 aa, Medicago truncatula
MKKVEADKCGAYCPYPRLYCSGDCDCEPFIASLPPRLNFKCVTPHSSAELKKKVEEQPKLCWSHTECTEKGSGNYCARFPNSNLKYGLCFPSISEAVNTFKMASSLKFEKDFLKMSLPA

SEQ ID NO: 11 - protein, 127 aa, Medicago truncatula
MPTKVIFPMKKVEADKCGAYCPYPRLYCSGDCDCEPFIASLPPRLNFKCVTPHSSAELKKKVEEQPKLCWSHTECTEKGSGNYCARFPNSNLKYGLCFPSISEAVNTFKMASSLKFEKDFLKMSLPA

SEQ ID NO: 12 - protein, 146 aa, Trifolium medium
MTYAKLAPLAVFLLATFLMFPTKNVEACGGLCSVFDSRPVCGGGGCTCMFYWYAPVMGECQSIHNAMRQSINDTMVEESPYLCNSHADCTIKGSGSFCARYPNNPYNVKYGWCFASKYEAEDYVRVVGSRYKFNKDLLKMPTSAEY

SEQ ID NO: 13 - protein, 138 aa, Cicer arietinum
MAYVKLPPLTLFLLATFLITFSTKKVGATCTGVCSFYDSNPCGGNYCFCHFLDVGDYKGICIDRSFFIKTVDEDPNLCQTHAECTKKGSGNFCGRYPNPNIKYGRCFASNTEAEEFSNKFSYYSSRFIKDFLNMPVVA

SEQ ID NO: 14 - protein, 172 aa, Arachis hypogaea
MAKLTILVALALFLLAAHASARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 15 - protein, 149 aa, Fagopyrum esculentum
MKLFIILATATLLIAATQATYPRDEGFDLGETQMSSKCMRQVKMNEPHLKKCNRYIAMDILDDKYAEALSRVEGEGCKSEESCMRGCCVAMKEMDDECVCEWMKMMVENQKGRIGERLIKEGVRDLKELPSKCGLSELECGSRGNRYFV

SEQ ID NO: 16 - protein, 145 aa, Sinapis alba
PAGPFRIPKCRKEFQQAQHLRACQQWLHKQAMQSGSGPSWTLDDEFDFEDDMENPQGPQQRPPLLQQCCNELHQEEPLCVCPTLKGASKAVKQQVRQQLGQQGQQGPHLQHVISRIYQTATHLPKVCNIRQVSVCPFKKTMPGPS

SEQ ID NO: 17 - protein, 158 aa, Glycine max
MTKFTILLISLLFCIAHTCSASKWQHQQDSCRKQLQGVNLTPCEKHIMEKIQGRGDDDDDDDDDNHILRTMRGRINYIRRNEGKDEDEEEEGHMQKCCTEMSELRSPKCQCKALQKIMENQSEELEEKQKKKMEKELINLATMCRFGPMIQCDLSSDD

SEQ ID NO: 18 - protein, 146 aa, Bertholletia excelsa
MAKISVAAAALLVLMALGHATAFRATVTTTVVEEENQEECREQMQRQQMLSHCRMYMRQQMEESPYQTMPRRGMEPHMSECCEQLEGMDESCRCEGLRMMMMRMQQEEMQPRGEQMRRMMRLAENIPSRCNLSPMRCPMGGSIAGF

SEQ ID NO: 19 - protein, 155 aa, Capparis masaikai
MAKLIFLFATLALFVLLANASIQTTVIEVDEEEDNQLWRCQRQFLQHQRLRACQRFIHRRAQFGGQPDELEDEVEDDNDDENQPRRPALRQCCNQLRQVDRPCVCPVLRQAAQQVLQRQIIQGPQQLRRLFDAARNLPNICNIPNIGACPFRAWP

SEQ ID NO: 20 - protein, 130 aa, Sinapis arvensis
PAGPFRLPRCRKEFQQAQHLRACQQWLHKQARQSGSGPSPQGPQQRPPLLQQCCNELHQEEPLCVCPTLKGAAKAVKQQIQQQGQQHGQQGQQLQHEIRRIYQTATHLPKVCNIPQVQVCPFNKTMPGPS

SEQ ID NO: 21 - protein, 148 aa, Sesamum indicum
MARFTIVLAVLFAAALVSASAHKTVVTTSVAEEGEEENQRGCEWESRQCQMRHCMQWMRSMRGQYEESFLRSAEANQGQFEHFRECCNELRDVKSHCRCEALRCMMRQMQQEYGMEQEMQQMQQMMQYLPRMCGMSYPTECRMRPIFA

SEQ ID NO: 22 - protein, 110 aa, Brassica napus
QPQKCQREFQQEQHLRACQQWIRQQLAGSPFQSGPQEGPWLREQCCNELYQEDQVCVCPTLKQAAKSVRVQGQHGPFQSTRIYQIAKNLPNVCNMKQIGTCPFIAIPFFP

SEQ ID NO: 23 - protein, 141 aa, Helianthus annuus
MARFSIVFAAAGVLLLVAMAPVSEASTTTIITTIIEENPYGRGRTESGCYQQMEEAEMLNHCGMYLMKNLGERSQVSPRMREEDHKQLCCMQLKNLDEKCMCPAIMMMLNEPMWIRMRDQVMSMAHNLPIECNLMSQPCQM

SEQ ID NO: 24 - protein, 165 aa, Arabidopsis thaliana
MAKLILVFATLALFILLANASIYRTVVEFEEDDDVSNPQQGKCQREFMKHQQLRGCKQWIRKRAQQGRIGYEADDFELTLDVDLEDDENPMGPQQQSSLKMCCNELRQVDKMCVCPTLKKAAQQVRFQGMHGQQQVQHVFQTAKNLPNVCKIPTVGSCQFKASPY

SEQ ID NO: 25 - protein, 164 aa, Arabidopsis thaliana
MANKLFLVCATLALCFLLTNASIYRTVVEFEEDDASNPVGPRQRCQKEFQQSQHLRACQRWMSKQMRQGRGGGPSLDDEFDFEGPQQGYQLLQQCCNELRQEEPVCVCPTLKQAARAVSLQGQHGPFQSRKIYQSAKYLPNICKIQQVGECPFQTTIPFFPPYY

SEQ ID NO: 26 - protein, 164 aa, Arabidopsis thaliana
MANKLFLVCAALALCFLLTNASIYRTVVEFEEDDATNPIGPKMRKCRKEFQKEQHLRACQQLMLQQARQGRSDEFDFEDDMENPQGQQQEQQLFQQCCNELRQEEPDCVCPTLKQAAKAVRLQGQHQPMQVRKIYQTAKHLPNVCDIPQVDVCPFNIPSFPSFY

SEQ ID NO: 27 - protein, 145 aa, Arachis hypogaea
MAKSTILVALLALVLVAHASAMRRERGRQGDSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVSGGRC

SEQ ID NO: 28 - protein, 152 aa, Lupinus angustifolius
MAKLTILIALVAALVLVVHTSAFRSSEQSCKRQLQQVNLRHCENHIDQRIQQQQEEEEDRARKLRGIKHVILRHKSSQESEESEELDQCCEQLNELNSQRCQCRALQQIYESQSEQCEGRQQEQQLEGELEKLPRICGFGPLRRCNINPDEE

SEQ ID NO: 29 - protein, 186 aa, Brassica napus
MANKLFLVSATLALFFLLTNASVYRTVVEVDEDDATNPAGPFRIPKCRKEFQQAQHLRACQQWLHKQAMQPGGGSGPSWTLDGEFDFEDDVENQQQGPQQRPPPPQQCCNELHQEEPLCVCPTLKGASKAVRQQVRQQQGQQMQGQQMQQVISRVYQTATHLPRVCNIRQVSICPFQKTMPGPGFY

SEQ ID NO: 30 - protein, 166 aa, Arabidopsis thaliana
MANKLFLVCAALALCFILTNASVYRTVVEFDEDDASNPIGPIQKCQKEFQQDQHLRACQRWMRKQMWQGRGGGPSLDDEFDMEDDIENPQRRQLLQKCCSELRQEEPVCVCPTLRQAAKAVRFQGQQHQPEQVRKIYQAAKYLPNICKIQQVGVCPFQIPSIPSYY

SEQ ID NO: 31 - protein, 170 aa, Arabidopsis thaliana
MANKLFLVCATFALCFLLTNASIYRTVVEFDEDDASNPMGPRQKCQKEFQQSQHLRACQKLMRMQMRQGRGGGPSLDDEFDLEDDIENPQGPQQGHQILQQCCSELRQEEPVCVCPTLRQAARAVSLQGQHGPFQSRKIYKTAKYLPNICKIQQVGECPFQTTIPFFPPY

SEQ ID NO: 32 - protein, 180 aa, Brassica napus
MANKLFLVSATLAFFFLLTNASIYRTIVEVDEDDATNPAGPFRIPKCRKEFQQAQHLKACQQWLHKQAMQSGSGPSWTLDGEFDFEDDMENPQGPQQRPPLLQQCCNELHQEEPLCVCPTLKGASKAVKQQVRQQQGQQGQQLQQVISRIYQTATHLPKVCNIPQVSVCPFQKTMPGPSY

SEQ ID NO: 33 - protein, 178 aa, Brassica napus
MANKLFLVSATLAFFFLLTNASIYRTVVEFDEDDATNPAGPFRIPKCRKEFQQAQHLKACQQWLHKQAMQSGSGPSWTLDGEFDFEDDMENPQGPQQRPPLLQQCCNELHQEEPLCVCPTLKGASKAVKQQIQQQGQQQGKLQMVSRIYQTATHLPKVCKIPQVSVCPFQKTMPGPSY

SEQ ID NO: 34 - protein, 154 aa, Bertholletia excelsa
MAKMSVVAAALLALLVLGQATAFRTTVTTTLEEEQEENPRGRSEQQCREQMERQQQLNHCRMYLRQQMEESPYQNPRPLRRGEEPHLDECCEQLERMDEMCRCEGLRMMLRRQREEMELQGEQMQRIMRKAENLLSRCNLSPQRCPMGGYTAWL

SEQ ID NO: 35 - protein, 178 aa, Brassica napus
MANKLFLVSATLAFFFLLTNASIYRTVVEFDEDDATDSAGPFRIPKCRKEFQQAQHLRACQQWLHKQAMQSGGGPSWTLDGEFDFEDDMENPQGPQQRPPLLQQCCNELHQEEPLCVCPTLKGASKAVKQQIQQQGQQQGKQQMVSRIYQTATHLPKVCNIPQVSVCPFQKTMPGPSY

SEQ ID NO: 36 - protein, 129 aa, Brassica juncea
AGPFRFPRCRKEFQQAQHLRACQQWLHKQAMQSGSGPQPQGPQQRPPLLQQCCNELHQEEPLCVCPTLKGASKAVKQQIRQQGQQQGQQGQQLQHEISRIYQTATHLPRVCNIPRVSICPFQKTMPGPS

SEQ ID NO: 37 - protein, 104 aa, Capparis masaikai
EPLCRRQFQQHQHLRACQRYLRRRAQRGGLADEQRGPALRLCCNQLRQVNKPCVCPVLRQAAHQQLYQGQIEGPRQVRRLFRAARNLPNICKIPAVGRCQFTRW

SEQ ID NO: 38 - protein, 104 aa, Capparis masaikai
EPLCRRQFQQHQHLRACQRYIRRRAQRGGLVDEQRGPALRLCCNQLRQVNKPCVCPVLRQAAHQQLYQGQIEGPRQVRQLFRAARNLPNICKIPAVGRCQFTRW

SEQ ID NO: 39 - protein, 133 aa, Brassica napus
PKCRKEFQQAQHLKACQQWLHKQAMQSGGGPSWTLDGEFDFEDDMEKQGPQQRPPLHQQYCNELQQEEPLCVCPTLRGASKAVKQQIQQQEQQQGKQQMVNRIYQTATHLPKVCNIPQVSVCPFQKTMPGPSY

SEQ ID NO: 40 - protein, 153 aa, Lupinus angustifolius
MAKLTILIALVAALVLVVHTSAFQSSKQSCKRQLQQVNLRHCENHIAQRIQQQQEEEEDHALKLRGIKHVILRHRSSQEYSEESEELDQCCEQLNELNSQRCQCRALQQIYESQSEQCEGSQQEQQLEQELEKLPRTCGFGPLRRCDVNPDEE

SEQ ID NO: 41 - protein, 125 aa, Brassica napus
SAGPFRIPKCRKEFQQAQHLRACQQWLHKQAMQSGSGPQGPQQRPPLLQQCCNELHQEEPLCVCPTLKGASRAVKQQVRQQQGQQGQQLQQVISRIYQTATHLPKVCNIPQVSVCPFQKTMPGPS

SEQ ID NO: 42 - protein, 100 aa, Capparis masaikai
EPLCRRQFQQHQHLRACQRYLRRRAQRGEQRGPALRLCCNQLRQVNKPCVCPVLRQAAHQQLYQGQIEGPRQVRRLFRAARNLPNICKIPAVGRCQFTRW

SEQ ID NO: 43 - protein, 149 aa, Lupinus angustifolius
MAKLTILIALVAALVLVVHTSAFRSSEQSCKRQLQQVNLRHCENHIDQRIQQQQEEEEDRARKLRGIKHVILRHKSSQESEELDQCCEQLNELNSQRCQCRALQQIYESQSEQCEGRQQEQQLEGELEKLPRICGFGPLRRCNINPDEE